(19) United States
(12) Patent Application Publication
(10) Pub. No.: US 2013/0171142 A1
(43) Pub. Date: Jul. 4, 2013

(54) METHODS OF USING ANTI-CD3 ANTIBODIES TO PREVENT WEIGHT GAIN

(71) Applicants: Aoife Brennan, Dover, MA (US); Charlotte Mckee, Newton, MA (US); Lou Vaickus, Hingham, MA (US); Douglas Ringler, Boston, MA (US)

(72) Inventors: Aoife Brennan, Dover, MA (US); Charlotte Mckee, Newton, MA (US); Lou Vaickus, Hingham, MA (US); Douglas Ringler, Boston, MA (US)

(21) Appl. No.: 13/502,625

(22) PCT Filed: Oct. 20, 2010
(86) PCT No.: PCT/US10/53433

§ 371 Date: Mar. 18, 2013

Related U.S. Application Data
(60) Provisional application No. 61/253,474, filed on Oct. 20, 2009.

(51) Int. Cl. A61K 39/395

(52) U.S. Cl. CPC A61K 39/3955
USPC 424/135.1; 424/173.1; 424/133.1

(57) ABSTRACT

Provided herein are methods of administering anti-CD3 antibodies or antigen binding fragments to a human for decreasing weight gain or increasing weight loss. In certain embodiments, the human exhibits a body mass index (BMI) or greater than or equal to about 27. In certain embodiments, the anti-CD3 antibody or antigen binding fragment does not bind or has reduced binding to at least one class of Fc (gamma) receptor.

### RELATED APPLICATIONS

The present application claims the benefit of U.S. Provisional Patent Application Ser. No. 61/253,474, filed Oct. 20, 2009, which is herein incorporated by reference in its entirety.

### TECHNICAL FIELD

Provided herein are methods for reducing weight gain or increasing weight loss by administering anti-CD3 antibodies or antigen binding fragments thereof to an animal.

### BACKGROUND

CD3 is part of a functional T cell receptor (TCR) complex found on the surface membranes of T lymphocytes. This complex is referred to interchangeably herein as the CD3/TCR complex or the CD3/TCR complex. In mammals, CD3 is a protein complex composed of several distinct polypeptide chains: a CD3-gamma chain, a CD3-delta chain, two CD3-epsilon chains, and two CD3-zeta chains. These chains associate with either an alpha/beta or a gamma/delta TCR complex to generate a functional CD3/TCR complex. Binding of a CD3/TCR complex to a peptide antigen presented on a MHC molecule leads to transduction of a signal (e.g., an activating signal, a suppressive signal, or an inactivating signal) from the CD3/TCR complex to the metabolic machinery of the relevant T cell.

Antibodies against the CD3 molecule have been tested for efficacy in the treatment of certain diseases in humans. A number of these diseases, e.g., diabetes, are associated with, caused and/or exacerbated by obesity. Downstream aspects of obesity include, without limitation, low grade inflammation, insulin resistance, lipid abnormalities, chronic inflammatory state, abdominal obesity, and cardiovascular disease. Thus, methods of administering anti-CD3 antibodies and fragments thereof that reduce weight gain or increase weight loss would be advantageous.

### SUMMARY

Provided herein are methods of administering anti-CD3 antibodies or antigen binding fragments to a human for decreasing weight gain or increasing weight loss. In certain embodiments, the human exhibits a body mass index (BMI) of greater than or equal to about 27. In certain embodiments, the anti-CD3 antibody or antigen binding fragment does not bind or has reduced binding to at least one class of Fc (gamma) receptor.

In certain embodiments, methods disclosed herein comprise administering an anti-CD3 antibody or antigen binding fragment thereof to a selected human with a body mass index greater than or equal to about 27, e.g., greater than or equal to about 30, greater than or equal to about 32, or greater than or equal to about 34.

In certain embodiments, the human gains less weight or loses more weight after or during the dosing regimen than would be observed in a human with a BMI less than 27 when administered an equivalent amount of the antibody or antigen binding fragment according to the dosing regimen. In certain embodiments, the human gains less weight or loses more weight after or during the dosing regimen than would be observed in a human with a BMI greater than or equal to about 27 when the human is not administered the anti-CD3 antibody or antigen binding fragment according to the dosing regimen. In certain embodiments, the human gains less weight or loses more weight at the end of twelve months.

In certain embodiments, the human suffers from an immune-related disease, e.g., type I diabetes, type II diabetes, psoriasis, rheumatoid arthritis, lupus, inflammatory bowel disease, ulcerative colitis, Crohn's disease, Graves' thyroiditis, Graves' ophthalmopathy, multiple sclerosis, metabolic syndrome, effects form organ transplantation, or graft-versus-host disease (GVHD). In certain embodiments, the human does not suffer from an immune-related disease.

In certain embodiments, the anti-CD3 antigen binding fragment is selected from the group consisting of a Fab fragment, a F(ab′)₂ fragment and a scFv fragment.

In certain embodiments, the anti-CD3 antibody or antigen binding fragment is chimeric. In certain embodiments, the anti-CD3 antibody or antigen binding fragment is humanized. In certain embodiments, the anti-CD3 antibody or antigen binding fragment comprises an Fc domain, wherein the Fc domain is aglycosylated.

In certain embodiments, the anti-CD3 antibody or antigen binding fragment comprises an amino acid sequence of SEQ ID NO: 3, SEQ ID NO: 4, or both. In certain embodiments, the anti-CD3 antibody or antigen binding fragment comprises an alanine at an amino acid position corresponding to amino acid position 299 of SEQ ID NO: 1. In certain embodiments, the anti-CD3 antibody is selected from the group consisting of hOKT3, hOKT3γ1(Ala-Ala), HUM291, and NI-0401.

In certain embodiments, methods disclosed herein comprise administering an anti-CD3 antibody or antigen binding fragment, both of which do not bind or have reduced binding to at least one class of Fc (gamma) receptor compared to the OKT3 antibody, e.g., at least 50% reduced binding. In certain embodiments, methods disclosed herein comprise administering an anti-CD3 antibody or antigen binding fragment, both of which do not bind or have reduced binding to at least one class of Fc (gamma) receptor compared to the IgG1 antibody produced by the ARH-77 cell line deposited under ATCC catalog number CRL-1621, e.g., at least 50% reduced binding. In certain embodiments, the anti-CD3 antibody or antigen binding fragment is administered over a dosing regimen of at least five days or at least eight days.

In certain embodiments, the anti-CD3 antibody or antigen binding fragment thereof is administered on day one of the dosing regimen, and the amount of the anti-CD3 antibody or antigen binding fragment administered on each of days one and two does not exceed 0.5 mg per day, e.g., does not exceed 0.2 mg per day or 0.3 mg per day. In certain embodiments, the amount of the anti-CD3 antibody or antigen binding fragment administered on day one is about 0.1 mg, about 0.2 mg, or about 0.3 mg.

In certain embodiments, the amount of the anti-CD3 antibody or antigen binding fragment thereof administered on day three of the dosing regimen is less than about 0.5 mg greater than the amount of the anti-CD3 antibody or antigen binding fragment administered on day two, e.g., about 0.1 mg greater or about 0.2 mg greater. In certain embodiments, the amount of the anti-CD3 antibody or antigen binding fragment thereof administered on day four is less than about 0.55 mg greater than the amount of the anti-CD3 antibody or antigen binding fragment administered on day three, e.g., about 0.4 mg greater or about 0.45 mg greater. In certain embodiments, the amount of the anti-CD3 antibody or antigen binding fragment administered on day five is less than about 0.6 mg greater than the amount of the anti-CD3 antibody or antigen binding fragment thereof administered on day four, e.g., about 0.25 mg greater or about 0.4 mg greater. In certain embodiments, the amount of the anti-CD3 antibody or antigen binding fragment administered on day five is more than 0.3 mg greater than the amount of the anti-CD3 antibody or antigen binding fragment administered on day two, e.g., more than about 0.75 mg greater or more than about 1.0 mg greater. In certain embodiments, the amount of the anti-CD3 antibody or antigen binding fragment administered on day five is at least about 0.5 mg.

In certain embodiments, the amount of the anti-CD3 antibody or antigen binding fragment thereof administered is about 0.1 mg on day one, about 0.2 mg on day two, about 0.3 mg on day three, and about 0.75 mg on each of days four through eight. In certain embodiments, the amount of the anti-CD3 antibody or antigen binding fragment administered is about 0.1 mg on day one, about 0.2 mg on day two, about 0.3 mg on day 3, about 0.75 mg on day four, about 1.0 mg on day five, about 1.25 mg on day six, about 1.5 mg on day seven, and about 1.75 mg on day eight. In certain embodiments, the amount of the anti-CD3 antibody or antigen binding fragment administered is about 0.1 mg on day one; about 0.2 mg on day two, about 0.3 mg on day 3, about 0.75 mg on day four, about 1.0 mg on day five, about 1.25 mg on day six, about 1.5 mg on day seven, and about 3.75 mg on day eight. In certain embodiments, the amount of the anti-CD3 antibody or antigen binding fragment administered is about 0.2 mg on day one; about 0.4 mg on day two, about 0.6 mg on day 3, about 0.8 mg on day four, and about 1.1 mg on day five.

In certain embodiments, the anti-CD3 antibody or antigen binding fragment thereof is administered over a dosing regimen comprising at least four ramp days. In certain embodiments, the anti-CD3 antibody or antigen binding fragment is administered in an amount greater than about 0.1 mg and less than about 0.5 mg on ramp day one. In certain embodiments, the amount of the anti-CD3 antibody or antigen binding fragment administered on ramp day two is less than about 0.5 mg greater than the amount of the anti-CD3 antibody or antigen binding fragment administered on ramp day one, e.g., about 0.1 mg greater or about 0.2 mg greater. In certain embodiments, the amount of the anti-CD3 antibody or antigen binding fragment administered on ramp day three is less than about 0.55 mg greater than the amount of the anti-CD3 antibody or antigen binding fragment administered on ramp day two, e.g., about 0.4 mg greater or about 0.45 mg greater. In certain embodiments, the amount of the anti-CD3 antibody or antigen binding fragment administered on ramp day four is less than about 0.6 mg greater than the amount of the anti-CD3 antibody or antigen binding fragment administered on ramp day three, e.g., about 0.25 mg greater or about 0.4 mg greater. In certain embodiments, the amount of the anti-CD3 antibody or antigen binding fragment administered on ramp day four is more than 0.3 mg greater than the amount of the anti-CD3 antibody or antigen binding fragment administered on ramp day one, e.g., more than about 0.75 mg greater or more than about 1.0 mg greater. In certain embodiments, the amount of the anti-CD3 antibody or antigen binding fragment administered at least one ramp day is at least about 0.5 mg.

In certain embodiments, the anti-CD3 antibody or antigen binding fragment is administered on at least one pre-ramp day prior to ramp day one. In certain embodiments, the amount of the anti-CD3 antibody or antigen binding fragment administered on the at least one pre-ramp day does not exceed 0.3 mg or does not exceed 0.2 mg. In certain embodiments, the amount of the anti-CD3 antibody or antigen binding fragment administered on the at least one pre-ramp day is about 0.1 mg, about 0.2 mg, or about 0.3 mg.

In certain embodiments, the total amount of the antibody or antigen binding fragment administered is no greater than about 8.6 mg, no greater than about 6.85 mg, or no greater than about 3.1 mg. In certain embodiments, the anti-CD3 antibody or antigen binding fragment is administered intravenously.

In certain embodiments, the anti-CD3 antibody or antigen binding fragment is administered in a single daily dose on at least one day of the dosing regimen, e.g. on each day of the dosing regimen. In certain embodiments, the anti-CD3 antibody or antigen binding fragment is administered more than once a day on at least one day of the dosing regimen, e.g., on each day of the dosing regimen. In certain embodiments, the interval between administrations is at least one hour. In certain embodiments, the anti-CD3 antibody or antigen binding fragment is administered over a period of time on at least one day of the dosing regimen, e.g., over a period of at least fifteen minutes.

In certain embodiments, the anti-CD3 antibody or antigen binding fragment is administered with a pharmaceutically acceptable carrier or diluent. In certain embodiments, the anti-CD3 antibody or antigen binding fragment is administered in conjunction with another therapeutic agent, e.g., a weight loss agent.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF CERTAIN EMBODIMENTS

Provided herein are methods of administering anti-CD3 antibodies or antigen binding fragments to a human for decreasing weight gain or increasing weight loss. In certain embodiments, the human exhibits a body mass index (BMI) or greater than or equal to about 27. In certain embodiments, the anti-CD3 antibody or antigen binding fragment does not bind or has reduced binding to at least one class of Fc (gamma) receptor.

### DEFINITIONS

“Antibody” as the term is used herein refers to a protein that generally comprises heavy chain polypeptides and light chain polypeptides. IgG, IgD, and IgE antibodies comprise two heavy chain polypeptides and two light chain polypeptides. IgA antibodies comprise two or four of each chain and IgM generally comprises 10 of each chain. Single domain antibodies having one heavy chain and one light chain and heavy chain antibodies devoid of light chains are also contemplated. A given antibody comprises one of five types of heavy chains, called alpha, delta, epsilon, gamma and mu, the categorization of which is based on the amino acid sequence of the heavy chain constant region. These different types of heavy chains give rise to five classes of antibodies, IgA (including IgA1 and IgA2), IgD, IgE, IgG (IgG 1, IgG2, IgG3 and IgG4) and IgM, respectively. A given antibody also comprises one of two types of light chains, called kappa or lambda, the categorization of which is based on the amino acid sequence of the light chain constant domains.

“Antigen binding antibody fragment”, “antigen binding fragment”, or “fragment” as the terms are used herein refer to an antigen binding molecule that is not an antibody as defined above, but that has at least one antigen binding site of an antibody. Thus an antigen binding antibody fragment or antigen binding fragment of an anti-CD3 antibody is a fragment of an antibody that binds to CD3. Antigen binding fragments often comprise a cleaved portion of a whole antibody, although the term is not limited to such cleaved fragments. Antigen binding fragments can include, for example, Fab fragments, F(ab′) 2 fragments, scFv fragments, diabodies, linear antibodies, multispecific antigen binding fragments such as bispecific, trispecific, and multispecific antibodies (e.g., diabodies, triabodies, tetrabodies), minibodies, chelating recombinant antibodies, tribodies or bibodies, intrabodies, nanobodies, small modular immunopharmaceuticals (SMIP), binding-domain immunoglobulin fusion proteins, camelized antibodies, and V_{HH} containing antibodies.

“Humanized antibody” as the term is used herein refers to an antibody that has been engineered to comprise one or more human framework regions in the variable region together with non-human (e.g., mouse, rat, or hamster) complementarity-determining regions (CDRs) of the heavy and/or light chain. In certain embodiments, a humanized antibody comprises sequences that are entirely human except for the CDR regions. Humanized antibodies are typically less immunogenic to humans, relative to non-humanized antibodies, and thus offer therapeutic benefits in certain situations. Those of ordinary skill in the art will be aware of humanized antibodies, and will also be aware of suitable techniques for their generation.

“Chimeric antibody” as the term is used herein refers to an antibody that has been engineered to comprise a human constant region. Chimeric antibodies are typically less immunogenic to humans, relative to non-chimeric antibodies, and thus offer therapeutic benefits in certain situations. Those of ordinary skill in the art will be aware of chimeric antibodies, and will also be aware of suitable techniques for their generation.

“Dosing regimen,” “regimen” and “antibody dosing regimen,” as the terms are used herein, refers to the total course of treatment administered to an animal, e.g., treatment with an anti-CD3 antibody or antigen binding fragment thereof. In some embodiments, the total amount of the anti-CD3 antibody or antigen binding fragment administered to the patient does not exceed 300 μg/kg when administered intravenously, and when administered other than intravenously, the total amount administered does not exceed the bioequivalent of intravenous administration of 300 μg/kg.

A dosing regimen may include a given number of days of treatment. For example, an anti-CD3 dosing regimen may include administering an anti-CD3 antibody to an animal for a minimum number of days, a maximum number of days, or a specific number of days. As non-limiting examples, an anti-CD3 antibody may be administered to an animal over a regimen of five days, eight days, or any number of days in between or beyond. An anti-CD3 dosing regimen may be as short as one day, although as will be apparent from the remainder of the present specification, multiple day dosing regimens permit administration of higher amounts of antibody on later days while significantly reducing cytokine release syndrome and other negative effects. Regimens are generally 21 days or less (e.g., 18 days or less, 14 days or less, 12 days or less, 10 days or less, 8 days or less, 5 days or less, 3 days or less, 2 days or less, or 1 day) in length. Regimens can be separated by relatively short periods of time (e.g., 5 days, 10 days, 15 days, 20 days, 25 days, 30 days, 1.5 months, 2 months, 3 months, or 4 months) or longer periods of time (e.g., 6 months, 9 months, 12 months, 18 months. 2 years, 3 years, 4 years, 5 years, 10 years, 15 years, or 20 years). Additionally and/or alternatively, a regimen may include a given amount of therapeutic agent administered per day. For example, an anti-CD3 antibody or antigen binding fragment may be administered to an animal in a minimum amount on one or more days of the regimen, in a maximum amount on one or more days of the regimen, or in a specific amount on one or more days of the regimen.

As used herein, the term “therapy window” refers to the time period starting on the first day of a dosing regimen and extending past the last day of the dosing regimen to the first time at which no anti-CD3 antibody or antigen binding fragment thereof is detectable (using a standard ELISA assay) in the peripheral blood plasma of the human undergoing the relevant dosing regimen.

As used herein, the term “continuous” in the context of the time in which the mean level of free CD3/TCR complexes on appropriate T cells is within a specific range of levels, means that the time the mean level is in that specific range is not interrupted by any time in which that mean level is not within that specific range of levels.

As used herein, the term “not continuous” in the context of the time in which the mean level of free CD3/TCR complexes on appropriate T cells is within a specific range of levels, means that the time the mean level is in that specific range is interrupted by some amount of time (e.g., 15 minutes, 20 minutes, 30 minutes, 45 minutes, 1 hour, 2 hours, 3 hours, 4, hours, 5 hours, 6 hours, 8 hours, 10 hours, 12 hours, 14 hours, 16 hours 18 hours, 20 hours, 24 hours 28 hours, 32 hours, 36 hours, 40 hours, 44 hours, 48 hours, 60 hours, 72 hours, 84 hours, 90 hours, or any range of time of having upper and lower limits of any of above the specifically stated times), in which that mean level is not within that specific range of levels.

As used herein, the term “weight loss agent” refers to all pharmacological agents that reduce or control (i.e., prevent increases in) weight. Such agents can alter weight regulation, for example, by altering appetite, metabolism, or absorption of calories. Examples of suitable weight loss agents include without limitation, sibutramine and other serotonin and norepinephrine reuptake inhibitors (e.g., lorcaserin), orlistat (tetrahydrolipstatin), cetilistat, rimonabant, metformin, exenatide, and pramlintide. Within the meaning of the present disclosure, the term can also include non-pharmacological weight loss approaches such as diet, exercise regimens and/or surgery (e.g., gastric bypass surgery).

### Body Mass Index

“Body mass index” as the term is used herein refers to a number calculated from a person's weight and height according to the following formula: weight (kg)/[height (m)]². Body mass index (BMI) provides a reliable indicator of body fat for most people and is used to screen for weight categories that may lead to health problems. BMI does not measure body fat directly, but studies have shown that BMI correlates to several direct measurements of body fat. Thus, BMI is one tool for assessing whether a person is overweight or obese. Because determination of BMI requires only height and weight, it is inexpensive and easy to use for clinicians and for the general public. For adults, a BMI below 18.5 indicates the person is underweight. A BMI between 18.5 and 24.9 is considered normal. A BMI between 25.0 and 29.9 indicates that person is overweight. A BMI of 30.0 and above indicates that person is obese. Although BMI is calculated the same way for children and adults, since the amount of body fat changes with age, the criteria used to interpret the meaning of the BMI number for children and teens are different from those used for adults. Moreover, the amount of body fat differs between girls and boys. For these reasons, the interpretation of BMI is both age- and sex-specific for children and teens. Those of ordinary skill in the art will be aware of such age- and sex-specific criteria, and will be able to interpret calculated BMIs for children and teens accordingly.

In certain embodiments, methods disclosed herein comprise administering an anti-CD3 antibodies or antigen binding fragment thereof to a human according to a dosing regimen (e.g., any of the Exemplary Dosing Regimens or Ramped Dosing Regimens described below) for reducing weight gain or increasing weight loss. In certain embodiments, methods disclosed herein result in a subject gaining less weight than would otherwise be expected. In certain embodiments, methods disclosed herein result in a subject losing more weight than would otherwise be expected.

In certain embodiments, a human with a body mass index greater than or equal to about 27 (e.g., greater than or equal to about 27, greater than or equal to about 28, greater than or equal to about 29, greater than or equal to about 30, greater than or equal to about 31, greater than or equal to about 32, greater than or equal to about 33, greater than or equal to about 34, greater than or equal to about 35, or higher) gains less weight or loses more weight after or during the dosing regimen than would be observed in a human with a BMI less than about 27 when administered an equivalent amount of the antibody or antigen binding fragment according to the dosing regimen. For example, a human with a BMI or about 27 or more may gain less weight or lose more weight after or during the dosing regimen compared to a human with a BMI of less than 27, less than 26, less than 25, less than 24, less than 23, less than 22, less than 21, less than 20, less than 19, less than 18, or less.

In certain embodiments, a human with a body mass index greater than or equal to about 27 gains less weight or loses more weight after or during the dosing regimen than would be observed in a human with a BMI greater than or equal to about 27 when the human is not administered the anti-CD3 antibody or antigen binding fragment according to the dosing regimen. In certain embodiments, a human with a BMI of about 27 or higher (e.g., about 27, about 28, about 29, about 30, about 31, about 32, about 33, about 34, about 35, or higher) may gain less weight or lose more weight than a human with a similar or identical BMI. In certain embodiments, a human with a BMI of higher than about 27 (e.g., about 28, about 29, about 30, about 31, about 32, about 33, about 34, about 35, or higher) may gain less weight or lose more weight than a human with a lower BMI, but whose BMI is still above about 27. For example, a human with a BMI of about 30 may gain less weight or lose more weight than a human with a BMI of about 27.

In certain embodiments, a subject's weight is monitored and his or her BMI is calculated over a period of time following conclusion of the dosing regimen. For example, a subject's weight may be monitored and his or her BMI may be calculated for at least 8 weeks following conclusion of the dosing regimen, g., at least 8 weeks, at least 9 weeks, at least 10 weeks, at least 11 weeks, at least 12 weeks, at least 13 weeks, at least 14 weeks, at least 15 weeks, at least 16 weeks, or longer. In certain embodiments, a subject's weight may be monitored and his or her BMI may be calculated for at least 3 months following conclusion of the dosing regimen, e.g., at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, or longer. In certain embodiments, a subject's weight may be monitored and his or her BMI may be calculated for several years following conclusion of the dosing regimen.

### Fc Receptors

In certain embodiments, the anti-CD3 antibodies and antigen binding fragments thereof do not bind or have reduced binding to at least one class of Fc (gamma) receptor. The Fc receptors are a family of cell-surface molecules that bind the Fc portion of immunoglobulins. Each member of the family recognizes immunoglobulin of one isotype or a few closely related isotypes through a recognition domain on the alpha chain of the Fc receptor. Fc receptors are themselves members of the immunoglobulin superfamily. Different accessory cells bear Fc receptors for antibodies of different isotypes, and the isotype of the antibody thus determines which accessory cell will be engaged in a given response. There are at least four types of Fc receptor, including those belonging to the gamma (e.g., Fc (gamma) RI), epsilon (e.g., Fc (epsilon) RIa) and alpha (e.g., Fc (alpha) RI) groups, as well as the neonatal FcR (FcRn). FcRn transports IgG molecules across the placenta in humans and also across the gut in rats and mice. FcRn is also involved in the homeostasis of IgG in humans. Fc (epsilon) RI binds IgE with high affinity, Fc (alpha) RI binds IgA, and Fc (gamma) receptors bind IgG. The Fc (gamma) receptors are further divided into classes, which include at least Fc (gamma) RI, Fc (gamma) RII-A, Fc (gamma) RII-C, Fc (gamma) RII-B2, Fc (gamma) RII-B1, Fc (gamma) RIIIA, Fc (gamma) RIIIB, and Fc (gamma) RIV. These classes of Fc (gamma) receptors can vary in the types of cells on which they are expressed, the effects of their ligation (e.g., inhibitory or activating), and their affinity for the Fc of different antibody isotypes. For example, the affinity of Fc (gamma) RI for IgG1 is about 10⁸ M⁻¹; the affinities of Fc (gamma) RII-A, RII-B2 and RII-B1 for IgG1 are each about 2×10⁶ M⁻¹; and the affinity of Fc (gamma) RIII is about 5×10⁵ M⁻¹. A detailed description of Fc receptors is provided in Janeway, C. A. et al. Immunobiology; The Immune System in Health and Disease; (2001) 5th edition; Garland Publishing, New York, N.Y.; see, e.g., pages 362-363 and 370-377; and a detailed description of Fc (gamma) receptors is provided in Nimmerjahn and Ravetch; “Fcgamma receptors as regulator of immune responses”; Nat Rev Immunol. 2008 January; 8(1):34-47, the disclosures of which are incorporated herein by reference in their entireties.

### Exemplary Dosing Regimens

Provided herein are methods of administering anti-CD3 antibodies or antigen binding fragments thereof to a human (e.g., a human with a BMI greater than or equal to about 27) for reducing weight gain or increasing weight loss. In certain embodiments, the anti-CD3 antibody or antigen binding fragment to be administered does not bind or has reduced binding to at least one class of Fc (gamma) receptor. For example, the anti-CD3 antibody or antigen binding fragment may have reduced binding to at least one class of Fc (gamma) receptor as compared to the OKT3 antibody. As another example, the anti-CD3 antibody or antigen binding fragment may have reduced binding to at least one class of Fc (gamma) receptor as compared to the huOKT3-gamma-1 and/or huOKT3-gamma-1(A³¹⁸) antibodies as described in Xu et al., Cellular Immunology, 200, 16-26 (2000), incorporated herein by reference in its entirety. As another example, the anti-CD3 antibody or antigen binding fragment may have reduced binding to at least one class of Fc (gamma) receptor as compared to the IgG1 immunoglobulin produced by the ARH-77 cell line deposited under ATCC catalog number CRL-1621.

In certain embodiments, the anti-CD3 antibody or antigen binding fragment may be administered over a dosing regimen of one day, two days, three days, four days, five days, six days, seven days, eight days, nine days, ten days, eleven days, twelve days, thirteen days, fourteen days, or more. In certain embodiments, the anti-CD3 antibody or antigen binding fragment is administered over a dosing regimen of five days. In certain embodiments, the anti-CD3 antibody or antigen binding fragment is administered over a dosing regimen of eight days. In certain embodiments, the anti-CD3 antibody or antigen binding fragment is administered as a continuous infusion (e.g., by a microinfusion pump or slow-release patch) rather than a fixed dose. Limiting the number of days of a dosing regimen can confer practical benefits on a patient being treated. For example, limiting a dosing regimen to five days may minimize the inconvenience to a patient when that patient needs to travel to a hospital or clinic to receive anti-CD3 antibody or antigen binding fragment treatment. Limiting the number of days in a dosing regimen can also increase patient safety since fewer hospital visits will result in fewer medical recordkeeping requirements, and thus fewer chances of making recording or filing mistakes. Limiting the number of days in a given dosing regimen can also decrease the costs associated with treatment, since the treatment provider will need to spend less total time with the patient.

In certain embodiments, the anti-CD3 antibody or antigen binding fragment is administered on consecutive days during a given dosing regimen. In certain embodiments, the anti-CD3 antibody or antigen binding fragment is not administered on consecutive days of a dosing regimen. For example, a given dosing regimen may include one or more days in which the anti-CD3 antibody or antigen binding fragment is not administered. In certain embodiments, a dosing regimen comprises one, two, three, four, five, six, seven or more days in which an anti-CD3 antibody or antigen binding fragment is not administered. In certain embodiments, the anti-CD3 antibody or antigen binding fragment is administered every other day of a dosing regimen. In certain embodiments, the anti-CD3 antibody or antigen binding fragment is administered every third day, or every fourth day.

In certain embodiments, the anti-CD3 antibody or antigen binding fragment is administered in a low dose on at least one day of a dosing regimen. In certain embodiments, the anti-CD3 antibody or antigen binding fragment is administered in a low dose during the early portion of a dosing regimen, e.g., on the first one, two and/or three days of the regimen. As will be appreciated by those of ordinary skill in the art upon reading the present specification, administering the anti-CD3 antibody or antigen binding fragment in a low dose during the early portion of a dosing regimen facilitates the administration of higher individual doses later in a dosing regimen than would be possible with traditional dosing regimens. In certain embodiments, the anti-CD3 antibody or antigen binding fragment is administered in an amount that does not exceed about 0.5 mg per day during the early portion of a dosing regimen. For example, the anti-CD3 antibody or antigen binding fragment may be administered in an amount that does not exceed about 0.5 mg per day on the first one, two and/or three days of the regimen. In certain embodiments, the amount of the anti-CD3 antibody or antigen binding fragment administered on the first two days of the dosing regimen does not exceed about 0.5 mg per day. In certain embodiments, the amount of the anti-CD3 antibody or antigen binding fragment thereof administered on the first day of the dosing regimen does not exceed about 0.5 mg. In certain embodiments, the anti-CD3 antibody or antigen binding fragment thereof is administered in an amount that does not exceed about 0.45 mg per day, about 0.4 mg per day, about 0.35 mg per day, about 0.3 mg per day, about 0.25 mg per day, about 0.2 mg per day, about 0.15 mg per day, about 0.1 mg per day, about 0.09 mg per day, about 0.08 mg per day, about 0.07 mg per day, about 0.06 mg per day, about 0.05 mg per day, about 0.04 mg per day, about 0.03 mg per day, about 0.02 mg per day, about 0.01 mg per day, or less during the early portion of a dosing regimen, e.g. on the first one, two and/or three days of the regimen.

In certain embodiments, the amount of the anti-CD3 antibody or antigen binding fragment thereof administered on each of days one and two of a given dosing regimen does not exceed about 0.3 mg per day. In certain embodiments, the amount of the anti-CD3 antibody or antigen binding fragment thereof administered on each of days one and two of a given dosing regimen does not exceed about 0.2 mg per day. In certain embodiments, the amount of the anti-CD3 antibody or antigen binding fragment thereof administered on day one of a given dosing regimen is about 0.1 mg. In certain embodiments, the amount of the anti-CD3 antibody or antigen binding fragment thereof administered on day two of a given dosing regimen is about 0.2 mg. In certain embodiments, the amount of the anti-CD3 antibody or antigen binding fragment thereof administered on day two of a given dosing regimen is about 0.3 mg.

In certain embodiments, the amount of the anti-CD3 antibody or antigen binding fragment thereof administered increases between days two and five of a given dosing regimen. In certain embodiments, the amount of increase between days two and five is more than about 0.3 mg. For example, the amount of the anti-CD3 antibody or antigen binding fragment thereof administered may increase more than about 0.3 mg, more than about 0.35 mg, more than about 0.4 mg, more than about 0.45 mg, more than about 0.5 mg, more than about 0.55 mg, more than about 0.6 mg, more than about 0.65 mg, more than about 0.7 mg, more than about 0.75 mg, more than about 0.8 mg, more than about 0.85 mg, more than about 0.9 mg, more than about 0.95 mg, more than about 1.0 mg, more than about 1.1 mg, more than about 1.2 mg, more than about 1.3 mg, more than about 1.4 mg, more than about 1.5 mg, more than about 1.6 mg, more than about 1.7 mg, more than about 1.8 mg, more than about 1.9 mg, more than about 2 mg, more than about 2.5 mg, more than about 3 mg, more than about 3.5 mg, more than about 4 mg, more than about 4.5 mg, more than about 5 mg, or more.

In certain embodiments, the amount of the anti-CD3 antibody or antigen binding fragment thereof administered increases on each day between days two and five of a given dosing regimen such that the total increase between days two and five is more than about 0.3 mg. In certain embodiments, the amount of the anti-CD3 antibody or antigen binding fragment thereof administered between days two and five of a given dosing regimen increases by more than about 0.3 mg, but the amount of the anti-CD3 antibody or antigen binding fragment thereof administered does not increase on each day. For example, the amount of the antibody or antigen binding fragment thereof administered may remain constant or even decrease between, e.g., days two and three, days three and four, or days four and five, but the total amount nevertheless increases by more than about 0.3 mg between days two and five.

In certain embodiments, the amount of the anti-CD3 antibody or antigen binding fragment thereof administered on day three of a given dosing regimen is less than about 0.5 mg greater than the amount of the anti-CD3 antibody or antigen binding fragment thereof administered on day two of the dosing regimen. For example, the amount of the anti-CD3 antibody or antigen binding fragment thereof administered on day three of the dosing regimen may be less than about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater, or less than on day two. In certain embodiments, the amount of the anti-CD3 antibody or antigen binding fragment thereof administered on day three of the dosing regimen is about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater than on day two. In certain embodiments, the amount of the anti-CD3 antibody or antigen binding fragment thereof administered on day three of the dosing regimen is about equal to the amount administered on day two. In certain embodiments, the amount of the antibody or antigen binding fragment thereof administered on day three of the dosing regimen is less than the amount administered on day two. For example, the amount of the anti-CD3 antibody or antigen binding fragment thereof administered on day three of the dosing regimen may be about 0.01 mg less, about 0.02 mg less, about 0.03 mg less, about 0.04 mg less, about 0.05 mg less, about 0.06 mg less, about 0.07 mg less, about 0.08 mg less, about 0.09 mg less, about 0.1 mg less, about 0.15 mg less, about 0.2 mg less, about 0.25 mg less, about 0.3 mg less, about 0.35 mg less, about 0.4 mg less, about 0.45 mg less, about 0.5 mg less, than the amount administered on day two. In certain embodiments, the amount of the anti-CD3 antibody or antigen binding fragment thereof administered on day three of the dosing regimen is more than about 0.5 mg less than the amount administered on day two.

In certain embodiments, the amount of the anti-CD3 antibody or antigen binding fragment thereof administered on day four of a given dosing regimen is less than about 0.55 mg greater than the amount of the anti-CD3 antibody or antigen binding fragment thereof administered on day three of the dosing regimen. For example, the amount of the anti-CD3 antibody or antigen binding fragment thereof administered on day four of the dosing regimen may be less than about 0.55 mg greater, about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater, or less than on day three. In certain embodiments, the amount of the anti-CD3 antibody or antigen binding fragment thereof administered on day four of the dosing regimen is about 0.55 mg greater, about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater than on day three. In certain embodiments, the amount of the anti-CD3 antibody or antigen binding fragment thereof administered on day four of the dosing regimen is about equal to the amount administered on day three. For example, the amount of the anti-CD3 antibody or antigen binding fragment thereof administered on day four of the dosing regimen may be about 0.01 mg less, about 0.02 mg less, about 0.03 mg less, about 0.04 mg less, about 0.05 mg less, about 0.06 mg less, about 0.07 mg less, about 0.08 mg less, about 0.09 mg less, about 0.1 mg less, about 0.15 mg less, about 0.2 mg less, about 0.25 mg less, about 0.3 mg less, about 0.35 mg less, about 0.4 mg less, about 0.45 mg less, about 0.5 mg less, than the amount administered on day three. In certain embodiments, the amount of the anti-CD3 antibody or antigen binding fragment thereof administered on day four of the dosing regimen is more than about 0.5 mg less than the amount administered on day three.

In certain embodiments, the amount of the anti-CD3 antibody or antigen binding fragment thereof administered on day five of a given dosing regimen is less than about 0.6 mg greater than the amount of the anti-CD3 antibody or antigen binding fragment thereof administered on day four of the dosing regimen. For example, the amount of the anti-CD3 antibody or antigen binding fragment thereof administered on day five of the dosing regimen may be less than about 0.6 mg greater, about 0.55 mg greater, about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater, or less than on day four. In certain embodiments, the amount of the anti-CD3 antibody or antigen binding fragment thereof administered on day five of the dosing regimen is about 0.6 mg greater, about 0.55 mg greater, about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater than on day four. In certain embodiments, the amount of the anti-CD3 antibody or antigen binding fragment thereof administered on day five of the dosing regimen is about equal to the amount administered on day four. In certain embodiments, the amount of the anti-CD3 antibody or antigen binding fragment thereof administered on day five of the dosing regimen is less than the amount administered on day four. For example, the amount of the anti-CD3 antibody or antigen binding fragment thereof administered on day five of the dosing regimen may be about 0.01 mg less, about 0.02 mg less, about 0.03 mg less, about 0.04 mg less, about 0.05 mg less, about 0.06 mg less, about 0.07 mg less, about 0.08 mg less, about 0.09 mg less, about 0.1 mg less, about 0.15 mg less, about 0.2 mg less, about 0.25 mg less, about 0.3 mg less, about 0.35 mg less, about 0.4 mg less, about 0.45 mg less, about 0.5 mg less, than the amount administered on day four. In certain embodiments, the amount of the anti-CD3 antibody or antigen binding fragment thereof administered on day five of the dosing regimen is more than about 0.5 mg less than the amount administered on day four.

In certain embodiments, the amount of the anti-CD3 antibody or antigen binding fragment thereof administered on day five of a given dosing regimen is at least about 0.5 mg. For example, the amount of the anti-CD3 antibody or antigen binding fragment thereof administered on day five of a given dosing regimen can be at least about 0.5 mg, at least about 0.55 mg, at least about 0.6 mg, at least about 0.65 mg, at least about 0.7 mg, at least about 0.75 mg, at least about 0.8 mg, at least about 0.85 mg, at least about 0.9 mg, at least about 0.95 mg, at least about 1 mg, at least about 1.2 mg, at least about 1.3 mg, at least about 1.4 mg, at least about 1.5 mg, at least about 1.6 mg, at least about 1.7 mg, at least about 1.8 mg, at least about 1.9 mg, at least about 2 mg, at least about 2.5 mg, at least about 3 mg, at least about 3.5 mg, at least about 4 mg, at least about 4.5 mg, at least about 5 mg, or higher. In certain embodiments, the amount of the anti-CD3 antibody or antigen binding fragment thereof administered on day five of a given dosing regimen is about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, about 1 mg, about 1.2 mg, about 1.3 mg, about 1.4 mg, about 1.5 mg, about 1.6 mg, about 1.7 mg, about 1.8 mg, about 1.9 mg, about 2 mg, about 2.5 mg, about 3 mg, about 3.5 mg, about 4 mg, about 4.5 mg, about 5 mg, or higher.

In certain embodiments, an anti-CD3 antibody or antigen binding fragment thereof is administered according to the following dosing regimen: about 0.1 mg on day one, about 0.2 mg on day two, about 0.3 mg on day three, and about 0.5 mg on each of days four through eight. In certain embodiments, an anti-CD3 antibody or antigen binding fragment thereof is administered according to the following dosing regimen: about 0.1 mg on day one, about 0.2 mg on day two, about 0.3 mg on day three, and about 0.75 mg on each of days four through eight. In certain embodiments, an anti-CD3 antibody or antigen binding fragment thereof is administered according to the following dosing regimen: about 0.1 mg on day one, about 0.2 mg on day two, about 0.3 mg on day three, about 0.75 mg day four, about 1.0 mg on day five, about 1.25 mg on day six, about 1.5 mg on day seven, and about 1.75 mg on day eight. In certain embodiments, an anti-CD3 antibody or antigen binding fragment thereof is administered according to the following dosing regimen: about 0.1 mg on day one, about 0.2 mg on day two, about 0.3 mg on day three, about 0.75 mg day four, about 1.0 mg on day five, about 1.25 mg on day six, about 1.5 mg on day seven, and about 3.5 mg on day eight. In certain embodiments, an anti-CD3 antibody or antigen binding fragment thereof is administered according to the following dosing regimen: about 0.1 mg on day one, about 0.3 mg on day two, about 0.5 mg on day three, about 0.9 mg day four, and about 1.3 mg on day five. In certain embodiments, the anti-CD3 antibody or antigen binding fragment is administered according to the following dosing regimen: about 0.2 mg on day one, about 0.4 mg on day two, about 0.6 mg on day three, about 0.8 mg day four, and about 1.1 mg on day five. In certain embodiments, the anti-CD3 antibody or fragment is administered according to the following dosing regimen: about 0.2 mg on day one, about 0.4 mg on day two, about 0.8 mg on day three, about 1.4 mg on day four, and about 1.6 mg on day five. In certain embodiments, the anti-CD3 antibody or fragment is administered according to the following dosing regimen: about 0.1 mg on day one, about 0.3 mg on day two, about 0.6 mg on day three, about 1.2 mg on day four, and about 2.2 mg on day five.

In certain embodiments, the anti-CD3 antibody or antigen binding fragment is administered in multiple doses on one or more days of any of the above-described dosing regimens. For example, the anti-CD3 antibody or antigen binding fragment thereof may be administered in two doses on day eight of a given dosing regimen to achieve a total daily dose of 3.75 mg or more.

In certain embodiments, the total amount of the anti-CD3 antibody or antigen-binding fragment thereof administered to the patient does not exceed 300 μg/kg when administered intravenously, and when administered other than intravenously, the total amount administered does not exceed the bioequivalent of intravenous administration of 300 μg/kg.

In certain embodiments, the total amount of the anti-CD3 antibody or antigen binding fragment thereof administered over the course of a dosing regimen is no greater than about 21 mg. For example, the total amount of the anti-CD3 antibody or antigen binding fragment thereof administered to a patient over the course of a dosing regimen may no greater than about 21 mg, about 20 mg, about 19 mg, about 18 mg, about 17 mg, about 16 mg, about 15 mg, about 14 mg, about 13 mg, about 12 mg, about 11.5 mg, about 11 mg, about 10.5 mg, about 10 mg, about 9.5 mg, about 9 mg, about 8.5 mg, about 8 mg, about 7.5 mg, about 7 mg, about 6.5 mg, about 6 mg, about 5.5 mg, about 5 mg, about 4.5 mg, about 4 mg, about 3.9 mg, about 3.8 mg, about 3.7 mg, about 3.6 mg, about 3.5 mg, about 3.4 mg, about 3.3 mg, about 3.2 mg, about 3.1 mg, about 3 mg, about 2.9 mg, about 2.8 mg, about 2.7 mg, about 2.6 mg, about 2.5 mg, about 2.4 mg, about 2.3 mg, about 2.2 mg, about 2.1 mg, about 2 mg, about 1.9 mg, about 1.8 mg, about 1.7 mg, about 1.6 mg, about 1.5 mg, 1.4 mg, 1.3 mg, 1.2 mg, 1.1 mg, 1 mg, or less. In certain embodiments, the total amount of the anti-CD3 antibody or antigen binding fragment administered over the course of a dosing regimen is no greater than about 8.6 mg. In certain embodiments, the total amount of the anti-CD3 antibody or antigen binding fragment administered over the course of a dosing regimen is no greater than about 6.85 mg. In certain embodiments, the total amount of the anti-CD3 antibody or antigen binding fragment administered over the course of a dosing regimen is no greater than about 3.1 mg.

Any method of administration may be used to administer anti-CD3 antibodies or antigen binding fragments thereof to a patient. In certain embodiments, the anti-CD3 antibody or antigen binding fragment is administered to a patient intravenously. In certain embodiments, the anti-CD3 antibody or antigen binding fragment is administered to a patient by a route other than an intravenous route. For example, the anti-CD3 antibody or antigen binding fragment may be administered to a patient orally, rectally, intramuscularly, intranasally, subcutaneously, intraocularly, transdermally, by direct injection into an affected organ or tissue site, or inhaled. In certain embodiments, the anti-CD3 antibody or antigen binding fragment is administered as a continuous infusion (e.g., by a microinfusion pump or slow-release patch) rather than a fixed dose. In some embodiments, the patient self-administers the anti-CD3 antibody or antigen binding fragment. Those of ordinary skill in the art will be aware of suitable routes of administration and will be able to adapt such routes of administration to any of the dosing regimens disclosed herein.

In certain embodiments, the anti-CD3 antibody or antigen binding fragment is administered in a single daily dose on at least one day of a dosing regimen. In certain embodiments, the anti-CD3 antibody or antigen binding fragment is administered in a single daily dose on each day of a dosing regimen. A single daily dose of the anti-CD3 antibody or antigen binding fragment may be administered over a relatively short period of time, e.g., within a period of less than about fifteen minutes. Such embodiments minimize the hospital time and inconvenience to a patient. Alternatively, a single daily dose may be administered to a patient over a longer period of time, e.g., over a period of greater than fifteen minutes. For example, a single daily dose may be administered to a patient over a period of fifteen minutes, thirty minutes, forty-five minutes, one hour, two hours, three hours, four hours, five hours, six hours, seven hours, eight hours, nine hours, ten hours, eleven hours, twelve hours, or more. Such embodiments are useful when, for example, the patient experiences adverse side effects from administering the anti-CD3 antibody or antigen binding fragment over a relatively short period of time. Administration of an anti-CD3 antibody or antigen binding fragment to a patient over a period of time may be accomplished in any of a variety of ways such as, without limitation, intravenous administration.

In certain embodiments, the anti-CD3 antibody or antigen binding fragment is administered more than once a day on at least one day of a dosing regimen. In certain embodiments, the anti-CD3 antibody or antigen binding fragment is administered more than once a day on each day of a dosing regimen. For example, an antibody or antigen binding fragment can be administered twice, three times or four times on at least one day, or each day, of a dosing regimen. In such embodiments, there will typically be an interval between daily doses. For example, the interval between daily doses can be 1 hour, 2 hours, three hours, four hours, five hours, six hours, seven hours, eight hours, nine hours, ten hours, eleven hours, twelve hours, or more. Such embodiments are useful when, for example, the patient experiences adverse side effects from administration of the antibody or antigen binding fragment in a single daily dose.

In certain embodiments, methods disclosed herein can be used to decrease weight gain or increase weight loss in nonhuman animals. Although BMI is a parameter that is used only for humans, those of ordinary skill in the art will be able to select animals that exhibit certain weights, heights and or other body measurements that correlate with BMI, and will be able to apply the teachings in the present application to such animals. Moreover, doses and methods of administration may be selected in accordance with known principles of veterinary pharmacology and medicine. Guidance may be found, for example, in Adams, R. (ed.), Veterinary Pharmacology and Therapeutics, 8^{th} edition, Iowa State University Press; ISBN: 0813817439; 2001, the disclosure of which is hereby incorporated by reference in its entirety.

### Ramped Dosing Regimens

Provided herein are methods of administering anti-CD3 antibodies or antigen binding fragments thereof to a human (e.g., a human with a BMI greater than or equal to about 27) for reducing weight gain or increasing weight loss. Any of the dosing regimens disclosed herein, e.g., any of the dosing regimens disclosed in the “Exemplary Dosing Regimens” section above, may contain a ramping period. “Ramp” or “ramping period” as the terms are used herein refer to a portion of a dosing regimen over which the amount of the anti-CD3 antibody or antigen binding fragment administered increases from a ramp day at the beginning of the ramping period to a ramp day at the end of the ramping period. “Ramp day” as the term is used herein refers to a given day within the ramping period. In certain embodiments, the ramping period is at least two days, e.g., at least three days, at least four days, at least five days, at least six days, at least seven days, at least eight days, at least nine days, at least ten days, at least eleven days, at least twelve days, at least thirteen days, at least fourteen days, or more. In certain embodiments, the ramping period is at most fourteen days, e.g., at most thirteen days, at most twelve days, at most eleven days, at most ten days, at most nine days, at most eight days, at most seven days, at most six days, at most five days, at most four days, at most three days, or fewer. In certain embodiments, the ramping period is two days, three days, four days, five days, six days, seven days, eight days, nine days, ten days, eleven days, twelve days, thirteen days, fourteen days or more. In certain embodiments, the ramping period is four days. The ramp (or ramping period) does not include the first day of two or more days in which the dose of anti-CD3 antibody or antigen binding fragment administered is the same or decreases. In this case, the two or more days are non-ramp days and the non-ramp period consists of the two or more days. Thus, for example, in a regimen consisting of an anti-CD3 antibody or antigen binding fragment dosing schedule of 0.1 mg on day 1, 0.2 mg on day 2, 0.3 mg on day 3, 0.4 mg on day 0.4, 0.5 mg on day 6, 0.5 mg on day 7, and 0.5 mg on day 8, days 1-4 are ramp days, the ramp (or ramp period) consists of days 1-4, days 5-8 are non-ramp days, and the non-ramp period consists of days 5-7. It is understood that one or more ramps (ramp periods) can follow one or more non-ramp periods. The first day of a ramp is a day on which a dose that is administered is less than the immediately following dose. A pre-ramp day is a day prior to the first day of a ramp. Naturally, a pre-ramp day can be one or more days (e.g., 2 or more, 3 or more, 4 or more, 5 or more, 6 or more, 7, or more, 8 or more, nine or more, 10 or more, 11 or more, 12 or more, 13 or more, or 14 or more) before the first day of a ramp.

Methods disclosed herein that include a ramping period permit administration of higher cumulative doses of the anti-CD3 antibody or antigen binding fragment with decreased pro-inflammatory cytokine release and immunogenicity, and with minimal to no perturbation of Epstein Barr Virus immunity. In certain embodiments, methods disclosed herein that include a ramping period facilitate higher individual doses later in a dosing regimen than would be possible with traditional dosing regimens.

In general a ramping period comprises the following characteristics: the anti-CD3 antibody or antigen binding fragment is administered in an amount greater than about 0.1 mg and less than about 0.5 mg on ramp day one; the amount of the anti-CD3 antibody or antigen binding fragment administered on ramp day two is less than about 0.5 mg greater than the amount of the anti-CD3 antibody or antigen binding fragment administered on ramp day one; the amount of the anti-CD3 antibody or antigen binding fragment administered on ramp day three is less than about 0.55 mg greater than the amount of the anti-CD3 antibody or antigen binding fragment administered on ramp day two; the amount of the anti-CD3 antibody or antigen binding fragment administered on ramp day four is less than about 0.6 mg greater than the amount of the anti-CD3 antibody or antigen binding fragment administered on ramp day three; the amount of the anti-CD3 antibody or antigen binding fragment administered on ramp day four is more than 0.3 mg greater than the amount of the anti-CD3 antibody or antigen binding fragment administered on ramp day one; and the amount of the anti-CD3 antibody or antigen binding fragment administered at least one ramp day is at least about 0.5 mg.

In certain embodiments, the anti-CD3 antibody or antigen binding fragment is administered in an amount greater than about 0.1 mg and less than about 0.5 mg on ramp day one. For example, an anti-CD3 antibody or antigen binding fragment may be administered in an amount of about 0.1 mg, 0.15 mg, 0.2 mg, 0.25 mg, 0.3 mg, 0.35 mg, 0.4 mg, 0.45 mg, or 0.5 mg on ramp day one.

In certain embodiments, the amount of the anti-CD3 antibody or antigen binding fragment administered increases between ramp day one and ramp day four of a given dosing regimen. In certain embodiments, the amount of increase between ramp day one and ramp day four is more than about 0.3 mg. For example, the amount of the anti-CD3 antibody or antigen binding fragment administered may increase more than about 0.3 mg, more than about 0.35 mg, more than about 0.4 mg, more than about 0.45 mg, more than about 0.5 mg, more than about 0.55 mg, more than about 0.6 mg, more than about 0.65 mg, more than about 0.7 mg, more than about 0.75 mg, more than about 0.8 mg, more than about 0.85 mg, more than about 0.9 mg, more than about 0.95 mg, more than about 1.0 mg, more than about 1.1 mg, more than about 1.2 mg, more than about 1.3 mg, more than about 1.4 mg, more than about 1.5 mg, more than about 1.6 mg, more than about 1.7 mg, more than about 1.8 mg, more than about 1.9 mg, more than about 2 mg, more than about 2.5 mg, more than about 3 mg, more than about 3.5 mg, more than about 4 mg, more than about 4.5 mg, more than about 5 mg, or more.

In certain embodiments, the amount of the anti-CD3 antibody or antigen binding fragment administered increases on each day between ramp day one and ramp day four of a given dosing regimen such that the total increase between ramp day one and ramp day four is more than about 0.3 mg. In certain embodiments, the amount of the anti-CD3 antibody or antigen binding fragment administered between ramp day one and ramp day four of a given dosing regimen increases by more than about 0.3 mg, but the amount of the anti-CD3 antibody or antigen binding fragment administered does not increase on each day. For example, the amount of the anti-CD3 antibody or antigen binding fragment administered may remain constant or even decrease between, e.g., ramp day one and ramp day two, ramp day two and ramp day three, or ramp day three and ramp day four, but the total amount nevertheless increases by more than about 0.3 mg between ramp day one and ramp day four.

In certain embodiments, the amount of the anti-CD3 antibody or antigen binding fragment administered on ramp day two of a given dosing regimen is less than about 0.5 mg greater than the amount of the anti-CD3 antibody or antigen binding fragment administered on ramp day one of the dosing regimen. For example, the amount of the anti-CD3 antibody or antigen binding fragment administered on ramp day two of the dosing regimen may be less than about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater, or less than on ramp day one. In certain embodiments, the amount of the anti-CD3 antibody or antigen binding fragment administered on ramp day two of the dosing regimen is about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater than on ramp day one. In certain embodiments, the amount of the anti-CD3 antibody or antigen binding fragment administered on ramp day two of the dosing regimen is about equal to the amount administered on ramp day one. In certain embodiments, the amount of the anti-CD3 antibody or antigen binding fragment administered on ramp day two of the dosing regimen is less than the amount administered on ramp day one. For example, the amount of the anti-CD3 antibody or antigen binding fragment administered on ramp day two of the dosing regimen may be about 0.01 mg less, about 0.02 mg less, about 0.03 mg less, about 0.04 mg less, about 0.05 mg less, about 0.06 mg less, about 0.07 mg less, about 0.08 mg less, about 0.09 mg less, about 0.1 mg less, about 0.15 mg less, about 0.2 mg less, about 0.25 mg less, about 0.3 mg less, about 0.35 mg less, about 0.4 mg less, about 0.45 mg less, about 0.5 mg less, than the amount administered on ramp day one. In certain embodiments, the amount of the anti-CD3 antibody or antigen binding fragment administered on ramp day two of the dosing regimen is more than about 0.5 mg less than the amount administered on ramp day one.

In certain embodiments, the amount of the anti-CD3 antibody or antigen binding fragment administered on ramp day three of a given dosing regimen is less than about 0.55 mg greater than the amount of the anti-CD3 antibody or antigen binding fragment administered on ramp day two of the dosing regimen. For example, the amount of the anti-CD3 antibody or antigen binding fragment administered on ramp day three of the dosing regimen may be less than about 0.55 mg greater, about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater, or less than on ramp day two. In certain embodiments, the amount of the anti-CD3 antibody or antigen binding fragment administered on ramp day three of the dosing regimen is about 0.55 mg greater, about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater than on ramp day two. In certain embodiments, the amount of the anti-CD3 antibody or antigen binding fragment administered on ramp day three of the dosing regimen is about equal to the amount administered on ramp day two. For example, the amount of the anti-CD3 antibody or antigen binding fragment administered on ramp day three of the dosing regimen may be about 0.01 mg less, about 0.02 mg less, about 0.03 mg less, about 0.04 mg less, about 0.05 mg less, about 0.06 mg less, about 0.07 mg less, about 0.08 mg less, about 0.09 mg less, about 0.1 mg less, about 0.15 mg less, about 0.2 mg less, about 0.25 mg less, about 0.3 mg less, about 0.35 mg less, about 0.4 mg less, about 0.45 mg less, about 0.5 mg less, than the amount administered on ramp day two. In certain embodiments, the amount of the anti-CD3 antibody or antigen binding fragment administered on ramp day three of the dosing regimen is more than about 0.5 mg less than the amount administered on ramp day two.

In certain embodiments, the amount of the anti-CD3 antibody or antigen binding fragment administered on ramp day four of a given dosing regimen is less than about 0.6 mg greater than the amount of the anti-CD3 antibody or antigen binding fragment administered on ramp day three of the dosing regimen. For example, the amount of the anti-CD3 antibody or antigen binding fragment administered on ramp day four of the dosing regimen may be less than about 0.6 mg greater, about 0.55 mg greater, about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater, or less than on ramp day three. In certain embodiments, the amount of the anti-CD3 antibody or antigen binding fragment administered on ramp day four of the dosing regimen is about 0.6 mg greater, about 0.55 mg greater, about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater than on ramp day three. In certain embodiments, the amount of the anti-CD3 antibody or antigen binding fragment administered on ramp day four of the dosing regimen is about equal to the amount administered on ramp day three. In certain embodiments, the amount of the anti-CD3 antibody or antigen binding fragment administered on ramp day four of the dosing regimen is less than the amount administered on ramp day three. For example, the amount of the anti-CD3 antibody or antigen binding fragment administered on ramp day four of the dosing regimen may be about 0.01 mg less, about 0.02 mg less, about 0.03 mg less, about 0.04 mg less, about 0.05 mg less, about 0.06 mg less, about 0.07 mg less, about 0.08 mg less, about 0.09 mg less, about 0.1 mg less, about 0.15 mg less, about 0.2 mg less, about 0.25 mg less, about 0.3 mg less, about 0.35 mg less, about 0.4 mg less, about 0.45 mg less, about 0.5 mg less, than the amount administered on ramp day three. In certain embodiments, the amount of the anti-CD3 antibody or antigen binding fragment administered on ramp day four of the dosing regimen is more than about 0.5 mg less than the amount administered on ramp day three.

In certain embodiments, the amount of the anti-CD3 antibody or antigen binding fragment administered on ramp day four of a given dosing regimen is at least about 0.5 mg. For example, the amount of the anti-CD3 antibody or antigen binding fragment administered on ramp day four of a given dosing regimen can be at least about 0.5 mg, at least about 0.55 mg, at least about 0.6 mg, at least about 0.65 mg, at least about 0.7 mg, at least about 0.75 mg, at least about 0.8 mg, at least about 0.85 mg, at least about 0.9 mg, at least about 0.95 mg, at least about 1 mg, at least about 1.2 mg, at least about 1.3 mg, at least about 1.4 mg, at least about 1.5 mg, at least about 1.6 mg, at least about 1.7 mg, at least about 1.8 mg, at least about 1.9 mg, at least about 2 mg, at least about 2.5 mg, at least about 3 mg, at least about 3.5 mg, at least about 4 mg, at least about 4.5 mg, at least about 5 mg, or higher. In certain embodiments, the amount of the anti-CD3 antibody or antigen binding fragment administered on ramp day four of a given dosing regimen is about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, about 1 mg, about 1.2 mg, about 1.3 mg, about 1.4 mg, about 1.5 mg, about 1.6 mg, about 1.7 mg, about 1.8 mg, about 1.9 mg, about 2 mg, about 2.5 mg, about 3 mg, about 3.5 mg, about 4 mg, about 4.5 mg, about 5 mg, or higher.

In certain embodiments, the anti-CD3 antibody or antigen binding fragment is administered on at least one pre-ramp day prior to ramp day one. For example, an anti-CD3 antibody or antigen binding fragment may be administered on one, two, three, four, five, six, seven, eight, nine, ten, or more pre-ramp days prior to ramp day one. In certain embodiments, the amount of the anti-CD3 antibody or antigen binding fragment administered on at least one pre-ramp day does not exceed 0.3 mg, e.g., does not exceed 0.25 mg, 0.2 mg, 0.15 mg, 0.1 mg, 0.05 mg, or less. In certain embodiments, the amount of the anti-CD3 antibody or antigen binding fragment administered on at least one pre-ramp day is about 0.1 mg. In certain embodiments, the amount of the anti-CD3 antibody or antigen binding fragment administered on at least one pre-ramp day is about 0.2 mg. In certain embodiments, the amount of the anti-CD3 antibody or antigen binding fragment administered on at least one pre-ramp day is about 0.3 mg.

Dosing regimens Based on Body Weight and Body Surface Area

In certain embodiments, anti-CD3 antibodies or antigen binding fragments thereof can be administered without regard to body weight of the patient or to the body surface area of the patient (save that it will be recognized that the person to be administered the anti-CD3 antibody or antigen binding fragment will have a BMI of greater than or equal to about 27). For example, any of the dosing regimens described above can be administered to patient regardless of weight or body surface area.

In certain embodiments, anti-CD3 antibodies or antigen binding fragments thereof can be administered based on the body weight of the patient whose BMI is greater than or equal to about 27. Such body weight-based dosing regimens can also be useful when the subject is a juvenile and thus weighs significantly less than a typical adult patient. In certain embodiments, by calibrating the amount of antibody or antigen binding fragment administered based on the body weight of the patient, a more uniform amount of antibody or antigen binding fragment can be achieved across patients who differ in body weight.

Any dosing regimen, such as one of those as those described in the present specification, can be administered based on the body weight of the patient. A typical adult human has a body weight of between 70 and 80 kg, and dosing regimens described herein can be calculated on a per weight basis based on, for example, either of these weights. For example, if a non-weight-based dosing regimen calls for 0.1 mg of the anti-CD3 antibody or antigen binding fragment to be administered on particular day, a weight-based dose can be administered in an amount equal to 1.25 μg/kg (based on an 80 kg person in the original dosing regimen), or in an amount equal to 1.43 μg/kg (based on a 70 kg person in the original dosing regimen). Other daily doses of the anti-CD3 antibody or antigen binding fragment for a given dosing regimen can be similarly calculated. Thus, in certain embodiments, an adult with a higher body weight can receive a greater amount of the anti-CD3 antibody or antigen binding fragment, while adults with a lower body weight can receive a smaller amount of the anti-CD3 antibody or antigen binding fragment.

As another non-limiting example, the dosing regimen disclosed in Example 5 can be administered on the basis of a patient's weight. Example 5 discloses the following dosing schedule: 0.1 mg on day 1, 0.3 mg on day 2, 0.5 mg on day 3, 0.9 mg on day 4, and 1.3 mg on day 5. Based on a typical 80 kg patient, for example, one can administer the dosing schedule of Example 5 to a patient based on his or her specific body weight as follows: 1.25 μg/kg on day 1, 3.75 μg/kg on day 2, 6.25 μg/kg on day 3, 11.25 μg/kg on day 4, and 16.25 μg/kg on day 5. Based on a typical 70 kg patient, for example, one can administer the dosing schedule of Example 5 to a patient based on his or her specific body weight as follows: 1.43 μg/kg on day 1, 4.29 μg/kg on day 2, 7.15 μg/kg on day 3, 12.87 μg/kg on day 4, and 18.59 μg/kg on day 5. Those of ordinary skill in the art can calculate the amounts to be given based on any given body weight for any of the dosing regimens disclosed herein.

Juveniles have a significantly lower body weight than that of the typical adult. For example, a juvenile patient may have a body weight of 40 kg. In such a case, again taking a non-weight-based dosing regimen that calls for 0.1 mg of the anti-CD3 antibody or antigen binding fragment, the juvenile patient may be administered 50 μg (based on 1.25 μg/kg for an 80 kg adult) or 57.2 μg (based on 1.43 μg/kg for a 70 kg adult) of the anti-CD3 antibody or antigen binding fragment.

In certain embodiments, anti-CD3 antibodies or antigen binding fragments thereof can be administered based on the body surface area of the patient. Such body surface-based dosing regimens can be useful when, for example, the subject is significantly larger or smaller compared to a typical patient. Such body surface-based dosing regimens can also be useful when the subject is a juvenile whose body surface is significantly smaller than that of a typical adult patient. In certain embodiments, by calibrating the amount of antibody or antigen binding fragment administered based on the surface area of the patient, a more uniform amount of antibody or antigen binding fragment can be achieved across patients who differ in body surface areas.

Any dosing regimen, such as one of those described in the present specification, can be administered based on the body surface area of the patient. A typical adult human has a body surface area of approximately 1.7 square meters, and dosing regimens described herein can be calculated based on such a body surface area. For example, if a dosing regimen that is not based on body surface area calls for 0.1 mg of the anti-CD3 antibody or antigen binding fragment to be administered on particular day, a body surface-based dose can be administered in an amount equal to 58.82 μg/square meter (based on an average adult body surface of 1.7 square meters). Other daily doses of antibody or antigen binding fragment for a given dosing regimen can be similarly calculated. Thus, in certain embodiments, an adult with a larger body surface area can receive a greater amount of the antibody or antigen binding fragment, while adults with a smaller body surface area can receive a smaller amount of the antibody or antigen binding fragment.

As another non-limiting example, the dosing regimen disclosed in Example 5 can be administered on the basis of a patient's body surface area. Example 5 discloses the following dosing schedule: 0.2 mg on day 1, 0.4 mg on day 2, 0.6 mg on day 3, 0.8 mg on day 4, and 1.1 mg on day 5. Based on an average body surface area of 1.7 square meters per patient, for example, one can administer the dosing schedule of Example 5 to a patient based on his or her specific body surface area as follows: 117.65 μg/square meter on day 1, 235.29 μg/kg on day 2, 352.94 μg/kg on day 3, 470.59 μg/kg on day 4, and 647.06 μg/kg on day 5. Those of ordinary skill in the art can calculate the amounts to be given based on any given body surface area for any of the dosing regimens disclosed herein.

Juveniles have a lower body surface area than that of the typical adult. For example, a juvenile patient may have a body surface area of 1.3 square meters. In such a case, again taking a dosing regimen not based on body surface area that calls for 0.2 mg of the anti-CD3 antibody or antigen binding fragment, the juvenile patient may be administered 153.85 μg of the anti-CD3 antibody or antigen binding fragment.

Those of ordinary skill in the art will be able to calculate weight-based and body surface-based dosing regimens that correspond to any of the variety of dosing regimens disclosed in the present specification, and will be able to administer such dosing regimens to a patient.

Dosing regimens Based on Molecular Weight of Antibody or Antigen Binding Fragment

In certain embodiments, the anti-CD3 antibodies or antigen binding fragments thereof can be administered without regard to the molecular weight of the anti-CD3 antibody or antigen binding fragment, or to the number of antigen binding sites in a given anti-CD3 antibody or antigen binding fragment. For example, any of the dosing regimens described above can be administered to patient regardless of molecular weight or number of antigen binding sites.

“Molecular weight” is a term and concept well known to those of ordinary skill in the art. The molecular weight of a compound or composition is the weight of one molecule of the compound or composition, relative to the unified atomic mass unit u (defined as 1/12 the mass of one molecule of the carbon-12 isotope). A compound or composition having a given molecular weight can also be quantified by molar mass, which has a numerical value that is the average molecular weights of the molecules in the compound or composition multiplied by Avogadro's constant (approximately 6.022×10²³). Molar mass is expressed in terms of grams per mole.

Antibodies vary in molecular weight based on, for example, the length and amino acid composition of the heavy and light chain polypeptide sequences that make up the protein part of the antibody. Moreover, as is known to those of ordinary skill in the art, the molecular weight of an antibody varies according to the extent of post-translational modification the antibody undergoes. For example, antibodies are often subjected to glycosylation, in which one or more carbohydrate moieties is covalently attached to either the heavy or light chain polypeptide sequence. Even amongst a population of antibodies with identical heavy and light chain polypeptide sequences, the extent of glycosylation can vary. The molecular weights of many antibodies are known in the art. Additionally, the molecular weight of a particular antibody can be empirically determined with any of a variety of tools known to those of ordinary skill in the art such as, without limitation, mass spectrometry. Determining the molecular weight of any particular antibody is within the abilities of those of ordinary skill in the art.

Antigen binding fragments also vary in molecular weight based on, for example, the length and amino acid composition of the heavy and light chain polypeptide sequences and post-translational glycosylation patterns. Certain antigen binding fragments, such as without limitation, Fab fragment, F(ab′)₂ fragments, and scFv fragments, are typically of a much lower molecular weight that that of an antibody that includes both heavy and light polypeptide chains. As with full-length antibodies, the molecular weight of particular antigen binding fragment can be empirically determined with any of a variety of tools known to those of ordinary skill in the art such as, without limitation, mass spectrometry, and is within the abilities of those of ordinary skill in the art

In certain embodiments, anti-CD3 antibodies or antigen binding fragments thereof can be administered based on the molecular weight of that antibody or antigen binding fragment. Such molecular weight-based dosing regimens can be useful when, for example, a practitioner desires to administer a dosing regimen of a particular antibody or antigen binding fragment, the molecular weight of which differs from the molecular weight of another antibody or antigen binding fragment used in an identical or similar dosing regimen. In certain embodiments, by calibrating the amount of antibody or antigen binding fragment administered based on the molecular weight of the particular antibody or antigen binding fragment, a more uniform molar amount of antibody or antigen binding fragment can be administered to a patient.

For example, otelixizumab has an average molecular weight of approximately 145 kDa. Thus, if a particular dosing regimen calls for 0.1 mg of anti-CD3 antibody to be administered to a patient on a particular day, the patient can be administered approximately 6.90×10⁻¹⁰ moles of otelixizumab. Doses of different anti-CD3 antibodies or antigen binding fragments thereof can be similarly calculated based on the molecular weight of those antibodies or antigen binding fragments thereof. In certain embodiments, the anti-CD3 antibody or antigen binding fragment with a larger molecular weight is administered to the patient in a greater per-weight amount. In other embodiments, the anti-CD3 antibody or antigen binding fragment with a smaller molecular weight is administered to the patient in a lower per-weight amount.

As another non-limiting example, the dosing regimen disclosed in Example 5 can be administered based on the molecular weight of the anti-CD3 antibody or antigen binding fragment to be administered. Example 5 discloses the following dosing schedule: 0.2 mg on day 1, 0.4 mg on day 2, 0.6 mg on day 3, 0.8 mg on day 4, and 1.1 mg on day 5. Based on a reference antibody with a molecular weight of 145 kDa, for example, one can administer the dosing schedule of Example 5 to a patient based on the specific molecular weight of the anti-CD3 antibody of fragment to be administered as follows: 1.38×10⁻⁹ moles on day 1, 2.76×10⁻⁹ moles on day 2, 4.14×10⁻⁹ moles on day 3, 5.52×10⁻⁹ moles on day 4, and 7.59×10⁻⁹ moles on day 5. Those of ordinary skill in the art can calculate the molar amounts of antibody or antigen binding fragment to be given for any of the dosing regimens disclosed herein.

In certain embodiments, anti-CD3 antibodies or antigen binding fragments thereof can be administered based on the number of antigen binding sites present on the anti CD3 antibody or antigen binding fragment. As is known to those of ordinary skill in the art, a whole antibody includes two distinct antigen binding sites which are located in the hypervariable regions of the antibody. The antigen binding sites of whole antibodies are formed by an interaction between the variable regions of the heavy and light chains. Each antigen binding site is capable of binding one antigen. Thus, whole antibodies are capable of binding two antigens. Certain antigen binding fragments can also include two antigen binding sites. For example, a F(ab′)₂ fragment lacks the constant region of a whole antibody, yet retains two antigen binding sites. Certain antigen binding fragments include only a single antigen binding site. For example, Fab fragments and scFv fragments lack the constant region of a whole antibody, and include only a single antigen binding site. Those of ordinary skill in the art will be aware of various antigen binding fragments, and will know how many antigen binding sites each fragment contains.

In certain embodiments, anti-CD3 antibodies or antigen binding fragments thereof can be administered based on the number of antigen binding sites present in a given anti-CD3 antibody or antigen binding fragment. Such antigen binding site-based dosing regimens can be useful when, for example, a practitioner desires to administer a dosing regimen of a particular anti-CD3 antibody or antigen binding fragment that includes a different number of antigen binding sites as compared to the number of antigen binding sites of another anti-CD3 antibody or antigen binding fragment used in an identical or similar dosing regimen. In certain embodiments, by calibrating the amount of the anti-CD3 antibody or antigen binding fragment administered during a dosing regimen based on the number of antigen binding sites that the anti-CD3 antibody or antigen binding fragment possesses, a more uniform number of antigen binding sites can be administered to a patient.

For example, otelixizumab possesses two antigen binding sites per molecule. Thus, if a particular dosing regimen calls for 0.1 mg of antibody to be administered to a patient on a particular day, the patient can be administered approximately 0.1 mg of otelixizumab, or 0.2 mg of an anti-CD3 antibody or antigen binding fragment that possesses only one antigen binding site per molecule. Doses of different anti-CD3 antibodies or antigen binding fragments thereof can be similarly calculated based on the number of antigen binding sites those antibodies or antigen binding fragments thereof possess. In certain embodiments, an anti-CD3 antibody or antigen binding fragment with one antigen binding site per molecule is administered to the patient in a greater amount than an anti-CD3 antibody or antigen binding fragment with two or more antigen binding sites per molecule. In other embodiments, an anti-CD3 antibody or antigen binding fragment with two or more antigen binding sites per molecule is administered to the patient in a lower amount than an anti-CD3 antibody or antigen binding fragment with only one antigen binding site per molecule.

As another non-limiting example, the dosing regimen disclosed in Example 5 can be administered based on the number of antigen binding site the antibody or antigen binding fragment to be administered possesses. Example 5 discloses the following dosing schedule: 0.2 mg on day 1, 0.4 mg on day 2, 0.6 mg on day 3, 0.8 mg on day 4, and 1.1 mg on day 5. Based on a reference antibody having two antigen binding sites, for example, one can administer an anti-CD3 antibody or antigen binding fragment having only one antigen binding site to a patient according to the dosing schedule as follows: 0.4 mg on day 1, 0.8 mg on day 2, 1.2 mg on day 3, 1.6 mg on day 4, and 2.2 mg on day 5. Those of ordinary skill in the art can calculate the amount of antibody or antigen binding fragment to be given for any of the dosing regimens disclosed herein based on the number of antigen binding sites the antibody or antigen binding fragment possesses.

Those of ordinary skill in the art will be able to calculate weight-based and body surface-based dosing regimens that correspond to any of the variety of dosing regimens disclosed in the present specification, and will be able to administer such dosing regimens to a patient.

Moreover, those of ordinary skill in the art will be able to choose a dosing regimen of a particular anti-CD3 antibody or antigen binding fragment based on a combination of one or more of: the body weight of a patient, the body surface area of a patient, the molecular weight of the anti-CD3 antibody or antigen binding fragment, and the number of antigen binding sites of the anti-CD3 antibody or antigen binding fragment. For example, a patient that weighs more than 80 kg can be administered an anti-CD3 antibody or antigen binding fragment that possesses only one antigen binding site. In such an example, a larger amount of anti-CD3 antibody or antigen binding fragment can be administered to account for (1) the patient's increased weight, and (2) the fact that the anti-CD3 antibody or antigen binding fragment has fewer antigen binding sites than a bivalent whole antibody. Upon reading the present specification, those of ordinary skill in the art will be able to administer an anti-CD3 antibody or antigen binding fragment to a patient in a dosing regimen specifically tailored to the physical characteristics of the patient and/or the molecular properties of the anti-CD3 antibody or antigen binding fragment.

### PK/PD Parameters

Subjects administered any of the presently disclosed dosing regimens may experience one or more immunoregulatory effects, such as one or more of those described in this section, in addition to experiencing decreased weight gain and/or increased weight loss. The presently disclosed methods of decreasing weight gain or increasing weight loss are not limited in any way by any particular mechanism of action. Nevertheless, a number of pharmacodynamic (PD) effects of treating T cells with reduced Fc (gamma) receptor-binding anti-CD3 antibodies or CD3-binding fragments thereof according to methods disclosed herein, are observable. For convenience these reduced Fc (gamma) receptor-binding anti-CD3 antibodies and CD3-binding fragments are referred to in this PK/PD Parameters section as “CD3-binding agents.”

In broad terms, the immunoregulatory effects seen after administration of CD3-binding agents can be divided into two phases that can overlap to some degree. Thus in the initial early phase (from an hour up to about 14 days) following exposure of T cells (CD4+ and CD8+) to such CD3-binding agents (in vivo and in vitro) immunoregulatory effects that occur include down-modulation of CD3/TCR complexes on the surfaces of the T cells, induction of T cell anergy or hyporesponsiveness to antigen, induction of apoptosis of T cells, and a decrease in the numbers of T cells (CD4+T cells and CD8+T cells). With respect to in vitro exposures, solid or gel substrate (e.g., tissue culture well bottom or agarose bead)-bound anti-CD3 antibodies, and CD3-binding fragments thereof, that have reduced ability to bind Fc (gamma) receptors do not qualify as “CD3-binding agents” (as defined above) in this substrate-bound form since they act in the same way as anti-CD3 antibodies with normal, wild-type Fc (gamma) receptor binding activity in the presence of Fc (gamma) receptor expressing cell. In the later phase (from one day to 16 weeks or more) after the exposure, the levels of immunosuppressive CD4+T cells (Tregs) expressing both cell surface CD25 (i.e., CD25+) and the FoxP3 transcription factor (FoxP3+) are found to increase. Notably no increase in CD8+, CD25+, FoxP3+cells is seen. Some or all of these events are interrelated.

T cells that undergo apoptosis as a result of exposure to CD3-binding agents, which is generally by the Fas/Fas ligand pathway, are those that are activated by antigen prior to the exposure (and are progressing through the cell cycle) and are not resting T cells. T cells in the S-G2 phase of the cell cycle are particularly sensitive to this type of apoptosis. The decreases in the numbers of CD4+ and CD8+T cells that are seen in the first phase appear to reflect retrafficking of T cells (e.g., from the blood to lymphoid tissue and/or target organs) and, to a relatively small extent, the above-described apoptosis.

The initial decrease of antigen responsiveness of T cells that have not undergone apoptosis is to some degree correlated with CD3/TCR down-modulation on the surface of the T cells. Nevertheless, there are conditions under which drastically reduced antigen responsiveness in the T cells is observed in the face of significant levels of cell surface TCR (see, e.g., Schwartz (2003) Annu Rev. Immunol. 21:305-334, the disclosure of which is incorporated herein by reference in its entirety). These findings indicate that, while antigen hyporesponsiveness in the T cells exposed to CD3/TCR-binding agents is due at least in part to down-modulation of CD3/TCR complexes, it is likely also due to the other effects such as active CD3/TCR-mediated anergy induction. It is also clear that, while transient exposure of T cells to lower doses of CD3-binding agents results in transient anergy or antigen hyporesponsiveness of T cells and cell-surface CD3/TCR down-modulation (with full recovery within less than 24 hours of exposure), longer exposure to somewhat higher doses results in much longer, if not permanent, anergy or antigen hyporesponsiveness (see, e.g., Anasetti et al. (1990) J. Exp. Med. 172:1691-1700; and Forman et al. (2009) Immune Privilege and Tolerance-Therapeutic Antibody Approaches. In: Recombinant Antibodies for Immunotherapy, M. Little, Ed., Cambridge University Press, pp. 350-369, the disclosures of which are incorporated herein by reference in their entireties). Down-modulation of CD3/TCR in response to CD3-binding agents seems to be largely due to internalization of CD3-binding agent:CD3/TCR complexes rather than masking of the CD3/TCR complex by the binding agent.

The transient effects (anergy or antigen hyporesponsiveness of T cells and cell-surface CD3/TCR down-modulation) indicated above to occur as a result of exposure to CD3-binding agents are seen even when repeated doses (e.g., on a daily basis) are administered. The anergy/antigen hyporesponsiveness and cell-surface CD3/TCR down-modulation occur after the first administration but the levels of both return to normal (i.e., the levels prior to the first administration) by the time of the second administration. The same effect is seen after all subsequent administrations unless much higher doses are administered and/or the cells are exposed to the CD3-binding agent for a much longer time. This pattern of decrease and increase in these parameters is referred to herein as a “saw tooth pattern”. Interestingly, with respect to the levels of both CD4+ and CD8+T cells, while a saw tooth pattern is seen, it is accompanied by an overall decrease in the total numbers of the cells during the course of the CD3-binding agent (see, e.g., Examples 2-4). Thus, after each successive administration, the rebound seen after the initial decrease in cell numbers after an administration is to a lower level than after the immediately previous administration.

It is likely that the induction of anergy or antigen hyporesponsiveness in T cells by these CD3-binding agents that, as indicated above have reduced ability to bind to Fc (gamma) receptors, is analogous to that of altered peptide ligands (APL) (see, e.g.: Sloan-Lancaster et al. (1993) Nature 363:156-159; Sloan-Lancaster et al. (1994) Cell 79:913-922; and Madrenas et al. (1995) 267:515-518, the disclosures of which are incorporated herein by reference in their entirety) that result in weak or incomplete activation of T cells. One likely mechanism of CD3-binding agent-induced anergy induction involves reduction in the relative proportion of cell surface CD3/TCR multimeric clusters to cell-surface monovalent CD3/TCR complexes. It has been shown that CD3/TCR complexes on T cells occur as both monovalent units and multivalent cluster, the latter existing in a wide range multiplicities (from two to greater than 20 CD3/TCR monomers) and the monomer in each case containing a TCR α and β chain (or a TCR γ and δ chain) and one CD3δ, two CD3ε, one CD3γ, and two CD3ζ chains (see, e.g.: Alarcón et al. (2006) EMBO Reports 7: 490-495; and Schamel et al. (2005) J. Exp. Med. 202(4): 493-503, the disclosures of which are incorporated herein by reference in their entireties). Thus, by exposing T cells to increasing concentrations of CD3-binding agents, the relative level of higher avidity CD3/TCR multimer clusters is decreased, leaving behind the lower avidity CD3/TCR monovalent units and thereby reducing the potential CD3/TCR signal strength and T cell responsiveness. The lower the level of multimers left after exposure, the longer it will take a particular T cell to recover fully activating signal strength responsiveness by synthesizing new multimers and/or converting monomeric units into multivalent complexes. This phenomenon could also explain the “conditioning” effect observed when an animal (e.g., a human) is administered a dosing regimen that includes a ramping period, as disclosed herein. Without wishing to be bound by theory, it is hypothesized that conditioning may result from the lower ramping doses being sufficient to modulate but not activate, so that when subsequent larger activating doses are given later in a dosing regimen, the signal strength is weak or incomplete leading to relative low responses and anergy. At some critical concentration of CD3-binding agent and/or length of exposure of the T cell to the CD3-binding agent, the T cell will be rendered anergic for an extremely long time, possibly for its life time. The relative susceptibility of T cells to anergy induction would depend on a number of factors, including the relative number of multimeric CD3/TCR clusters to monovalent CD3/TCR units and the relative number of monomeric units in the clusters.

The induction of CD4+Tregs that occurs later in the response of CD4+T cells to CD3-binding agents is likely to be relatively more important in the long-term beneficial effects of CD3-binding agents to immune-related (especially T cell-mediate) diseases, including autoimmune diseases such as type I diabetes (insulin-dependent diabetes mellitus (IDDM)), psoriasis, multiple sclerosis, and rheumatoid arthritis. Their induction very likely involves factors (e.g., transforming growth factor β (TGF-β)) produced by, and/or cell-cell interactions with, the hyporesponsive (or completely anergized) T cells described above as well as antigen presenting cells such as dendritic cells, and does not necessarily require contacting of the Treg precursor cells themselves with a CD3-binding agent.

In light of the above considerations, methods of inducing hyporesponsiveness and/or anergy, apoptosis, decreases in the numbers of CD4+ and CD8+T cells, cell surface CD3/TCR down-modulation, and relative level of multivalent CD3/TCR clusters (as compared to monovalent CD3/TCR units) in target T cells (e.g., CD4+ and CD8+T cells to which the CD3-binding agents bind) down-modulation are provided. Also provided are methods for inducing CD4+, CD25+, FoxP3+Treg cells. All these methods involve exposing target T cells to CD3-binding agents either in vivo or in vitro. Where the exposing is in vitro, the CD3-binding agents are in solution rather than bound to a solid or gel substrate (see above). In the induction of Treg cells, the precursor of the Treg can be, but is not necessarily, a target T cell (as the term is used above). Moreover, CD3-binding agents can bind to established CD4+CD25+FoxP3+Tregs and thereby enhance their suppressive activity. The dosing and scheduling regimens and methods of administration for performing in vivo exposures can be any of those disclosed herein, as are the subjects to which the methods can be applied.

While the target T cells are more commonly CD4+T cells, it is understood that they can also be CD8+T cells. Moreover, CD4+ and CD8+effector T cells (e.g., pathogenic T cells involved in a disease process) are subject to the suppressive activity of CD4+CD25+FoxP3+Tregs. However, it is understood that CD25+, FoxP3+T regs per se are CD4+ and not CD8+. The CD3/TCR down-modulation can be complete (100%) or partial (e.g., at least or not greater than: 10%:20%; 30%; 40%; 50%; 60%; 70%; 80%; 90%; 95%; or 98%). The down-modulation of the number of multivalent CD3/TCR clusters (i.e., units containing more than one CD3/TCR complex unit (see above)) can be similarly complete or partial. An anergic T cell is one that has substantially no responsiveness (i.e., less than 5%) as compared to the responsiveness that T cell would have had without exposure to a CD3-binding agent or the average responsiveness of T cells having the same CD4/CD8 cell surface marker as well as other markers known in the art to be associated with pre-exposure, or lack thereof, to antigen. T cells can be naive T cells (i.e., those never pre-exposed to antigen), activated T cells (i.e., T cells exposed to antigen and displaying any of a variety of T cell activities, e.g., proliferation, cytotoxic activity, and cytokine production), or memory T cells (i.e., T cells exposed to antigen and having an enhanced ability to respond to the same antigen and not necessarily displaying an activated cell phenotype. Cell surface markers positively (+) and negatively (−) associated with naive T cells include: CD45RA+, CD26L+, CD45 edited isoforms (CD45RB, CD45RC, CD45RAB, CD45RAC, CD45RBC, CD45RO, CD45R (ABC))−, CD25−, CD44−, and CD69−. Cell surface markers positively (+) associated with activated T cells include: CD25+, CD69+, HLA-DR+, CD38+, and GITR+. Memory T cells fall into three broad categories, which are categorized as follows: central memory T cells (memory stem cells) (T_{CM}) (L-selectin+, chemokine receptor CCR7+, and produce interleukin (IL)-2 (IL-2) but not IL-4 or interferon γ (IFN-γ)); effector memory T cells (T_{EM}) and closely related effector memory T cells RA (T_{EMRA}) (L-selectin−, CCR7−, and produce IL-4 and IFN-γ).

With respect to pharmacokinetic (PK) data, it has been possible to determine PK parameters for a CD3-binding agent of interest (the TRX4 antibody, also known as otelixizumab) using data collected from a number of clinical studies (see Table 1). The serum otelixizumab concentrations versus time were described by a one-compartment model with Michaelis-Menten (MM) saturable elimination:

$$\frac{d {C}_{p}}{d t} = Input / {V}_{d} - {V}_{max} {C}_{p} / ( {K}_{m} + {C}_{p} )$$

$${C}_{p} ( 0 ) = 0$$

where Cₚ is serum concentration of otelixizumab, V_{d} is the volume of distribution, Vₘₐₓ is the capacity of the elimination process, and Kₘ is the affinity constant or the serum otelixizumab concentration at which the elimination rate attains one-half of Vₘₐₓ.

TABLE 1
Clinical Studies of Otelixizumab Included in PK Analysis

| | | | | Number of |
|---|---|---|---|---|
| Study | Group or Cohort | Doses (mg)^{a} | Disease ^{b} | subjects |
| I | Group A | 24, 8, 8, 8, 8, 8 | D | 3 |
| | Group B | 8, 8, 8, 8, 8, 8 | D | 37 |
| II | Cohort 1 | 1 | P | 4 |
| | Cohort 2 | 2 | P | 4 |
| | Cohort 3 | 4 | P | 8 |
| III | Cohort 1 | 0.1, 0.1, 0.1 | D | 4 |
| | Cohort 2 | 0.5, 0.5, 0.5 | D | 3 |
| | Cohort 9 | 0.1, 0.3, 0.5 | D | 4 |
| | Cohort 10 | 0.3, 0.5, 1.0 | D | 1 |
| | Cohort A | 0.1, 0.2, 0.3, 0.5 | D | 4 |
| | Cohort A(½) | 0.05, 0.1, 0.15, 0.25 | D | 1 |
| | Cohort B | 0.1, 0.2, 0.3, 0.75 | D | 4 |
| | Cohort C | 0.1, 0.2, 0.3, 1.0 | D | 1 |
| | CH1 | 0.1, 0.2, 0.3, 0.5 × 5 | D | 16 |
| | CH2 | 0.1, 0.2, 0.3, 0.75 × 5 | D | 18 |
| | CH3 | 0.1, 0.2, 0.3, 0.75, 1, | D | 6 |
| | | 1.25, 1.5, 1.75 | | |

^{a}Doses were given once daily for 1 to 8 days.
^{b} D—Type 1 diabetes; P—Psoriasis.

In Study I (Table 1), otelixizumab was administered 6 times. In Group A, otelixizumab concentrations remained more or less constant over the 6 days of dosing, whereas in group B they increased slightly, suggesting accumulation of the drug.

In Study II (Table 1), otelixizumab was administered only once. Extensive sampling was done over the 24 hours after drug administration. For the 1 mg and 2 mg doses, the concentrations decreased to below the LLQ (lower limit of quantification) in about 0.2 day. For the 4 mg dose, concentrations above LLQ were observed up to 0.8 day. A few subjects showed a biphasic decline with a very rapid first phase.

In Study III (Table 1), otelixizumab was administered daily for up to 8 days. Doses were substantially lower than in Studies I and II, and as a result, most (83%) concentrations were below the LLQ. Due to the limited amount of available PK data, simultaneous analysis of the PK and PD (pharmacodynamic) data was necessary to recover PK profiles. The model building process started with linear PK; however, the individual empirical Bayesian estimates of volume of distribution were dose-dependent, suggesting nonlinearity. Thus, MM elimination was used, leading to substantial improvement in the model. Such kinetic parameters were estimated Kₘ=0.968 μg/mL and Vₘₐₓ=1.35 μg/mL/day. At low concentrations, such as those observed in Study III, otelixizumab was eliminated linearly with elimination rate constant kₑₗ=Vₘₐₓ/Kₘ=1.39 day⁻¹. At high concentrations, elimination was saturated. The V_{d} was estimated as 13.9 L with between-subject variability of about 76%.

Biphasic elimination from serum is usually observed after an intravenous dose of intact antibodies. The intact antibodies rapidly distribute primarily to the highly perfused organs such as kidney, lung and liver. The volume of distribution often equals the plasma volume, 2-3 L. For otelixizumab, the V_{d} of 13.9 L was determined assuming a one-compartment model with MM elimination. This value of V_{d} suggests antibody distribution outside the blood or occurrence of nonspecific binding. Antigen binding can significantly affect the PK of a mAb. Target-mediated drug disposition models were proposed and successfully applied to describe the PK of certain mAbs. In the case of otelixizumab, elimination by binding to CD3/TCR complexes did not affect its PK. After otelixizumab administration, the CD3/TCR is down-modulated from T cell surfaces, and the transient trafficking and re-distribution of lymphocytes reduces the total pool of receptors available for binding. CD3/TCRThe MM elimination was used to approximate observed nonlinearities. The affinity constant (Kₘ=0.968 μg/mL) suggests that PK may become nonlinear at high concentrations such as those observed in Study I. For the dose ranges used in Study III, and to some degree in Study II, the drug is eliminated under linear conditions with a kₑᵢ of 1.39 dayl and a corresponding half-life of 0.50 day. Intact human IgG1 exhibits a long half-life of about 3 weeks due to the catabolic protection and recycling by the neonatal Fc receptor (FcRn). For otelixizumab the half-life is much shorter, suggesting that this protection pathway is not active, likely due to the single amino acid substitution in the Fc region which eliminates the only glycosylation site and alters the spatial configuration of the Fc region.

In view of the above PK considerations, in certain embodiments, the present disclosure provides a CD3-binding agent (see above) and a pharmaceutical composition containing it. The CD3-binding agent is an antibody (or CD3-binding fragment thereof) that binds to human CD3 with an affinity constant (Kₘ) of at least 0.968 μg/M1 and it can have a kₑₗ of about 1.39 day⁻¹. Moreover, its half life can be about 0.50 day when administered to a human.

The CD3-binding agent can show non-linear PK at high concentrations (about 8 mg to about 48 mg per day) and linear PK at low concentrations (about 0.1 to about 21 mg per day) Other features of the CD3-binding agent can be those described herein for otelixizumab (TRX4). Moreover the CD3-binding agent can be used in any of the methods and subjects described herein.

In certain embodiments, a pathogenic effect observed in the animal (e.g., on day five) or later of the dosing regimen is decreased or eliminated compared to the pathogenic effect that would be observed that day if the animal were administered a different dosing regimen. “Pathogenic effect” as the term is used herein refers to any adverse effect which results directly or indirectly from a given dosing regimen. A pathogenic effect may be, for example, increased cytokine release, (Epstein Barr Virus) EBV activation, or immunogenicity. In certain embodiments, the different dosing regimen lacks a ramping period. In certain embodiments, the different dosing regimen comprises a dose higher than 0.5 mg on either day one or day two of the different dosing regimen.

In certain embodiments, dosing regimens disclosed herein for decreasing weight gain or increasing weight loss result in a reduced level of release of at least one cytokine compared to an animal that is administered an equivalent dosing regimen of the anti-CD3 antibody or antigen binding fragment that does not exhibit reduced binding to the Fc (gamma) receptor. For example the release of the at least one cytokine may be reduced by at least 50%, e.g., at least 55%, at least 60%, at least 65%, at least 70%, at least 75%, at least 80%, at least 85%, at least 90%, at least 95%, at least 96%, at least 97%, at least 98%, at least 99%, or more. In certain embodiments, such a cytokine may be a pro-inflammatory cytokine including, but not limited to, IL2, IL6, IL10, IFN-gamma, and/or (tumor necrosis factor) TNF-alpha. In addition to cytokines, the dosing regimens of the present document may reduce the release of an enzyme such as tryptase in the same fashion recited above for cytokines Those of ordinary skill in the art will be aware of other pro-inflammatory cytokines, and will be able to measure their levels in a subject that has been administered any of the dosing regimens disclosed herein.

### Immune-Related Diseases

In certain embodiments, dosing regimens of the anti-CD3 antibody or antigen binding fragment disclosed herein can be administered to a human with a BMI greater than or equal to about 27 suffering from an immune-related disease. In such embodiments, the human exhibits less weight gain or increased weight loss compared to a human with a BMI less than 27, or compared to a human that does not receive the dosing regimen. “Immune-related disease” as the term is used herein refers to a disease that is associated with at least one abnormal immune phenomenon. For example, one class of immune-related diseases comprises autoimmune diseases. An autoimmune disease typically results when the subject's immune system is activated against one or more components (cells, tissues, or cell/tissue-free molecules) of the subject and attacks that subject's s own organs, tissues or cells, instead of attacking, for example, foreign bacteria, viruses and other infectious agents or cancer cells. Every mammalian subject exhibits autoimmunity to some extent, but such autoimmunity normally does not result in a disease state since the immune system regulates and suppresses normal autoimmunity. Autoimmune diseases develop when there is a disruption in the immune system's regulation. Autoimmune diseases can also result when there is a molecular alteration in a subject's cell that is recognized by the immune system, such that the immune system recognizes the altered cell as “foreign.”

Another example of an immune-related disease is a disease associated with the effects of organ, tissue, or cell transplantation. Cells transplanted into a subject rarely exhibit the same antigens on their surfaces as the subject's endogenous cells. Thus, a transplant subject's immune system often attacks and rejects the foreign cells. Certain immunosuppressive drugs are typically used to abrogate or decrease this immune attack, but such drugs often cause undesirable side effects, including for example, the risk of developing opportunistic infections as a result of decreased immune responses. In severe cases, an immune system attack on a transplanted organ can lead to organ failure or more serious systemic complications, such as, for example, graft-versus-host disease (GVHD), where the graft (e.g., bone marrow) includes immune-system effector cells (e.g., effector T cells) or precursors thereof.

Exemplary immune-related diseases include, but are not limited to, adrenergic drug resistance, alopecia greata, ankylosing spondylitis, antiphospholipid syndrome, autoimmune Addison's disease, autoimmune diseases of the adrenal gland, allergic encephalomyelitis, autoimmune hemolytic anemia, autoimmune hepatitis, autoimmune inflammatory eye disease, autoimmune neonatal thrombocytopenia, autoimmune neutropenia, autoimmune oophoritis and orchitis, autoimmune thrombocytopenia, autoimmune thyroiditis, Behcet's disease, bullous pemphigoid, cardiomyopathy, cardiotomy syndrome, celiac sprue-dermatitis, chronic active hepatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, dense deposit disease, diseases associated with effects from organ transplantation, discoid lupus, essential mixed cryoglobulinemia, fibromyalgia-fibromyositis, glomerulonephritis (e.g., IgA nephrophathy), gluten-sensitive enteropathy, Goodpasture's syndrome, graft-versus-host disease (GVHD), Graves' disease (including e.g., Graves' thyroiditis and Graves' ophthalmopathy), Guillain-Barre, hyperthyroidism (i.e., Hashimoto's thyroiditis), idiopathic pulmonary fibrosis, idiopathic Addison's disease, idiopathic thrombocytopenia purpura (ITP), IgA neuropathy, insulin resistance syndrome, juvenile arthritis, lichen planus, lupus erythematosus, Meniere's disease, metabolic syndrome, mixed connective tissue disease, multiple sclerosis, myasthenia gravis, myocarditis, diabetes (e.g., Type I diabetes or Type II diabetes), neuritis, other endocrine gland failure, pemphigus vulgaris, pernicious anemia, polyarteritis nodosa, polychrondritis, polyendocrinopathies, polyglandular syndromes, polymyalgia rheumatica, polymyositis and dermatomyositis, post-MI, primary agammaglobulinemia, primary biliary cirrhosis, psoriasis, psoriatic arthritis, Raynauld's phenomenon, relapsing polychondritis, Reiter's syndrome, rheumatic heart disease, rheumatoid arthritis, sarcoidosis, scleroderma, Sjogren's syndrome, stiff-man syndrome, systemic lupus erythematosus, takayasu arteritis, temporal arteritis/giant cell arteritis, ulcerative colitis, urticaria, uveitis, uveitis ophthalmia, vasculitides such as dermatitis herpetiformis vasculitis, vitiligo, and Wegener's granulomatosis.

Downstream aspects of obesity include, without limitation, low grade inflammation, insulin resistance, lipid abnormalities, chronic inflammatory state, abdominal obesity, and cardiovascular disease. In certain embodiments, any of the methods disclosed herein can be used to treat such downstream aspects.

### Anti-CD3 Antibodies and Antigen Binding Fragments Thereof

Any of a variety of the anti-CD3 antibodies or antigen binding fragments thereof can be employed in the dosing regimens described herein for decreasing weight gain or increasing weight loss. In certain embodiments, the anti-CD3 antibody or antigen binding fragment is a human antibody or antigen binding fragment. In certain embodiments, the anti-CD3 antibody or antigen binding fragment is a non-human antibody or antigen binding fragment, e.g., a mouse or rat anti-CD3 antibody or antigen binding fragment. In certain embodiments, the anti-CD3 antibody or antigen binding fragment is chimeric in that it contains human heavy and/or light chain constant regions. In certain embodiments, the anti-CD3 antibody or antigen binding fragment is humanized in that it contains one or more human framework regions in the variable region together with non-human (e.g., mouse, rat, or hamster) complementarity-determining regions (CDRs) of the heavy and/or light chain. In certain embodiments, the anti-CD3 antibody is monoclonal. In certain embodiments, the anti-CD3 antigen binding fragment is derived from a monoclonal antibody (e.g., cleaved at its hinge region to generate a F(ab′)₂ fragment). In certain embodiments, the anti-CD3 antibody is a polyclonal anti-CD3 antibody population in that it comprises a plurality of different antibodies, each of which binds to the same antigen. In certain embodiments, the antigen binding fragment is derived from a polyclonal anti-CD3 antibody population.

In certain embodiments, an antigen binding fragment is a Fab fragment, a F(ab′)₂ fragment, a scFv fragment, a diabody, a linear antibody, a multispecific antigen binding fragment such as a bispecific, a trispecific, or a multispecific antibody (e.g., a diabody, a triabody, a tetrabody), a minibody, a chelating recombinant antibody, a tribody or bibody, an intrabody, a nanobody, a small modular immunopharmaceutical (SMIP), a binding-domain immunoglobulin fusion protein, a camelid antibody, or a V_{HH} containing antibody.

In certain embodiments, the anti-CD3 antibody or antigen binding fragment to be employed in one or more of the dosing regimens disclosed herein binds a human CD3. A variety of anti-human CD3 antibodies and fragments are known in the art. Such anti-CD3 antibodies and fragments are useful, for example, when the animal to be treated is a human. In certain embodiments, the anti-CD3 antibody or antigen binding fragment to be employed in one or more of the dosing regimens disclosed herein binds a non-human CD3. For example, a non-human mammal may be administered the anti-CD3 antibody or antigen binding fragment, which anti-CD3 antibody or antigen binding fragment binds a CD3 present in that animal. Any of a variety of non-human mammals are known, and can be administered the anti-CD3 antibody or antigen binding fragment that binds a CD3 present in such that animal. Non-limiting examples include dogs, cats, cows, horses, sheep, goats, pigs, mice, rats, and hamsters. The anti-CD3 antibodies and antigen binding fragments can be of the same species or a different species. Moreover, they can be analogous to the chimeric and humanized antibodies described herein. Thus, when treating a horse, for example, the CD3 antibody or antigen binding fragment can contain heavy and/or light chain variable regions of another species (e.g., mouse, rat, hamster, or human) and horse heavy and/or light chain constant regions (chimeric heavy and/or light chains). Alternatively, heavy and/or light chains can contain all the CDRs from another species (as above) with the rest of the heavy and/or light chain being horse (horse analogs of humanized heavy and light chains). Moreover, the heavy chain or the light chain can be of the chimeric type and the other chain can be of the horse analog of the humanized chain. The same principles apply to anti-CD3 antibodies and antigen binding fragments for use in any of the exemplary species listed above.

In certain embodiments, the anti-CD3 antibody or antigen binding fragment to be employed in one or more of the dosing regimens disclosed herein binds a CD3 epsilon polypeptide, e.g., a human CD3 epsilon polypeptide. In certain embodiments, the anti-CD3 antibody or antigen binding fragment to be employed in one or more of the dosing regimens disclosed herein binds a CD3 gamma polypeptide, e.g., a human CD3 gamma polypeptide. In certain embodiments, the anti-CD3 antibody or antigen binding fragment to be employed in one or more of the dosing regimens disclosed herein binds a CD3 delta polypeptide, e.g., a human CD3 delta polypeptide. In certain embodiments, the anti-CD3 antibody or antigen binding fragment to be employed in one or more of the dosing regimens disclosed herein binds a CD3 zeta polypeptide, e.g., a human CD3 zeta polypeptide.

In certain embodiments, the anti-CD3 antibody or antigen binding fragment to be employed in one or more of the dosing regimens disclosed herein is otelixizumab, a humanized aglycosylated antibody. Otelixizumab, also known as TRX4, comprises a heavy chain having the sequence set forth in SEQ ID NO: 1 EVQLLESGGGLVQPGGSLRLSCAASGFTF SSFPMAWVRQAPGKGLEWVSTISTSGGRTYYRDSVKGRFTISRDNSKNTLYLQMNSLR AEDTAVYYCAKFRQYSGGFDYWGQGTLVTVSSASTKGPSVFPLAPSSKSTSGGTAALG CLVKDYFPEPVTVSWNSGALTSGVHTFPAVLQSSGLYSLSSVVTVPSSSLGTQTYICNVN HKPSNTKVDKKVEPKSCDKTHTCPPCPAPELLGGPSVFLFPPKPKDTLMISRTPEVTCVV VDVSHEDPEVIUNWYVDGVEVHNAKTKPREEQYASTYRVVSVLTVLHQDWLNGKEY KCKVSNKALPAPIEKTISKAKGQPREPQVYTLPPSRDELTKNQVSLTCLVKGFYPSDIAV EWESNGQPENNYKTTPPVLDSDGSFFLYSKLTVDKSRWQQGNVFSCSVMHEALHNHYT QKSLSLSPGK], and a light chain having the sequence set forth in SEQ ID NO: 2 [DIQLTQPNSVSTSLGSTVKLSCTLSSGNIENNYVHWYQLYEGRSPTTMIYDDDKRPDGV PDRFSGSIDRSSNSAFLTIHNVAIEDEAIYFCHSYVSSFNVFGGGTKLTVLRQPKAAPSVT LFPPSSEELQANKATLVCLISDFYPGAVTVAWKADSSPVKAGVETTTPSKQSNNKYAAS SYLSLTPEQWKSHRSYSCQVTHEGSTVEKTVAPTECS]. In certain embodiments, an anti-CD3 antibody or antigen binding fragment to be employed in one or more of the dosing regimens disclosed herein comprises the heavy chain variable region of otelixizumab, as set forth in SEQ ID NO: 3 [EVQLLESGGGLVQPGGSLRLSCAASGFTFSSFPMAWVRQAPGKGLEWVSTIS TSGGRTYYRDSVKGRFTISRDNSKNTLYLQMNSLRAEDTAVYYCAKFRQYSGGFDYW GQGTLVTVSS]. In certain embodiments, an anti-CD3 antibody or antigen binding fragment to be employed in one or more of the dosing regimens disclosed herein comprises the light chain variable region of otelixizumab, as set forth in SEQ ID NO: 4 [DIQLTQPN SVSTSLGSTVKLSCTLSSGNIENNYVHWYQLYEGRSPTTMIYDDDKRPDGVPDRFSGSID RSSNSAFLTIHNVAIEDEAIYFCHSYVSSFNVFGGGTKLTVLR].

In certain embodiments, an anti-CD3 antibody or antigen binding fragment to be employed in one or more of the dosing regimens disclosed herein comprises one or more complementarity determining regions (CDRs) of otelixizumab. For example, an anti-CD3 antibody or antigen binding fragment may include one or more of the following: the otelixizumab heavy chain variable complementarity determining region 1 (VH CDR1) comprising the amino acid sequence as set forth in SEQ ID NO: 5 [SFPMA], the otelixizumab heavy chain variable complementarity determining region 2 (VH CDR2) comprising the amino acid sequence as set forth in SEQ ID NO: 6 [TISTSGGRTYYRDSVKG], the otelixizumab heavy chain variable complementarity determining region 3 (VH CDR3) comprising the amino acid sequence as set forth in SEQ ID NO: 7 [FRQYSGGFDY], the otelixizumab light chain variable complementarity determining region 1 (VL CDR1) comprising the amino acid sequence as set forth in SEQ ID NO: 8 [TLSSGNIENNYVH], the otelixizumab light chain variable complementarity determining region 2 (VL CDR2) comprising the amino acid sequence as set forth in SEQ ID NO: 9 [DDDKRPD], or the otelixizumab light chain variable complementarity determining region 3 (VL CDR3) comprising the amino acid sequence as set forth in SEQ ID NO: 10 [HSYVSSFNV]. In certain embodiments, the anti-CD3 antibody or antigen binding fragment comprises each of the complementarity determining regions comprising the amino acid sequences set forth in SEQ ID NOs: 5-10.

The anti-CD3 antibody or antigen binding fragment thereof for use in the methods described herein can contain any combination of light and heavy chain, any combination of light and heavy chain variable regions, and any combination of light and heavy chain CDRs described above.

In certain embodiments, an anti-CD3 antibody or antigen binding fragment to be employed in one or more of the dosing regimens disclosed herein exhibits reduced binding to at least one class of Fc (gamma) receptor. In certain embodiments, binding of the modified anti-CD3 antibody or antigen binding fragment to at least one class of Fc (gamma) receptor is reduced as compared to the binding exhibited by the OKT3 antibody. OKT3 is a mouse antibody that is well-known to those of ordinary skill in the art. OKT3 binds the CD3 antigen, and is available from a variety of commercial sources (e.g., eBioscience™ at www.ebioscience.com). Additionally, a hybridoma cell line expressing the OKT3 antibody has been deposited under ATCC number CRL-8001. In certain embodiments an anti-CD3 antibody or antigen binding fragment to be employed in one or more of the dosing regimens disclosed herein exhibits at least 25% reduced binding to at least one class of Fc (gamma) receptor as compared to the binding that would be observed with the OKT3 antibody. For example, the anti-CD3 antibody or antigen binding fragment may exhibit at least 25%, 30%, 35%, 40%, 45%, 50%, 55%, 60%, 65%, 70%, 75%, 80%, 85%, 90%, 95%, 96%, 97%, 98%, 99% or more reduced binding.

In certain embodiments, binding of the modified anti-CD3 antibody or antigen binding fragment to at least one class of Fc (gamma) receptor is reduced as compared to the binding exhibited by the huOKT3-gamma-1 and/or huOKT3-gamma-1(A318) antibodies as described in Xu et al., Cellular Immunology, 200, 16-26 (2000), incorporated herein by reference in its entirety. In certain embodiments the anti-CD3 antibody or antigen binding fragment to be employed in one or more of the dosing regimens disclosed herein exhibits at least 25% reduced binding to at least one class of Fc (gamma) receptor as compared to the binding that would be observed with the huOKT3-gamma-1 and/or huOKT3-gamma-1(A318) antibodies. For example, the anti-CD3 antibody or antigen binding fragment may exhibit at least 25%, 30%, 35%, 40%, 45%, 50%, 55%, 60%, 65%, 70%, 75%, 80%, 85%, 90%, 95%, 96%, 97%, 98%, 99% or more reduced binding.

In certain embodiments, binding of the modified anti-CD3 antibody or antigen binding fragment to at least one class of Fc (gamma) receptor is reduced as compared to the binding exhibited by the IgG1 immunoglobulin molecule produced by the ARH-77 cell line deposited under ATCC catalog number CRL-1621. In certain embodiments the anti-CD3 antibody or antigen binding fragment to be employed in one or more of the dosing regimens disclosed herein exhibits at least 25% reduced binding to at least one class of Fc (gamma) receptor as compared to the binding that would be observed with the IgG1 antibody produced by the ARH-77 cell line deposited under ATCC catalog number CRL-1621. For example, the antibody or antigen binding fragment may exhibit at least 25%, 30%, 35%, 40%, 45%, 50%, 55%, 60%, 65%, 70%, 75%, 80%, 85%, 90%, 95%, 96%, 97%, 98%, 99% or more reduced binding.

In certain embodiments, an antibody or antigen binding fragment to be employed in one or more of the dosing regimens disclosed herein does not bind (e.g., exhibits no detectable binding) to at least one class of Fc (gamma) receptor.

In certain embodiments, an anti-CD3 antibody of fragment that exhibits reduced binding to at least one class of Fc (gamma) receptor comprises a modification that results in the reduced binding. In certain embodiments, such an antibody or antigen binding fragment may be modified at one or more amino acid residues within a heavy chain, a light chain, or both. The glycosylation state of an antibody or antigen binding fragment may affect its binding to one or more Fc (gamma) receptors. In certain embodiments, glycosylation of an anti-CD3 antibody or antigen binding fragment is altered by modifying one or more amino acid residues within a heavy chain, a light chain, or both. For example, otelixizumab comprises a human IgG1 heavy chain constant region that has been modified by replacing an asparagine at position 297 of SEQ ID NO: 1 with an alanine This modification results in decreased glycosylation of the antibody and significantly decreased binding of the antibody to major Fc receptors, compared to antibody molecules having wild type IgG1 constant regions, leading to decreased pro-inflammatory cytokine release and immunogenicity, and no perturbation of Epstein Barr Virus immunity. In certain embodiments, an anti-CD3 antibody or antigen binding fragment comprises an alanine at an amino acid position corresponding to amino acid position 299 of SEQ ID NO: 1. Position 299 of SEQ ID NO: 1 corresponds to amino acid residue number 297 of IgG heavy chains, according to the Kabat canonical numbering system (see Kabat E A, Wu T T, Perry H, Gottesman K, and Foeller C. (1991) Sequences of Proteins of Immunological Interest, Fifth Edition. NIH Publication No. 91-3242, incorporated herein by reference in its entirety). All IgG molecules contain a single conserved N-linked glycosylation site in each of their Cγ2 domains, which conserved glycosylation site corresponds to amino acid residue number 297 of IgG heavy chains, according to the Kabat canonical numbering system (see Arnold et al., The Impact of Glycosylation on the Biological Function and Structure of Human Immunoglobulins, Annu Rev. Immunol. 2007. 25:21-50, 2007, incorporated herein by reference in its entirety). Thus, in certain embodiments, such an IgG conserved glycosylation site is modified so as to reduce or eliminate glycosylation.

Other amino acid modifications of the anti-CD3 antibodies that result in reduced binding to at least one class of Fc (gamma) receptor are known in the art. For example, a humanized OTK3-derived antibody in which two amino acid residues at positions 234 and 235 of the Fc domain have been modified to alanine residues (referred to as hOKT3-gamma-1 (ala-ala)) is disclosed in United States Patent Publication numbers 2007/0077246 and 2008/0095766, the disclosures of which are incorporated herein by reference in their entireties. The hOKT3-gamma-1 (ala-ala) antibody is described as exhibiting reduced binding to Fc (gamma) receptors.

Other examples of the anti-CD3 antibodies include, without limitation, hOKT3 (humanized (IgG1 or IgG4) anti-human CD3), HUM291 (humanized (IgG2) anti-human CD3; visilizumab; NUVIONTM), UCHT1 (mouse (IgG1) anti-human CD3), Leu4 (mouse (IgG1) anti-human CD3), 500A2 (hamster (IgG) anti-mouse CD3), CLB-T3/3 (mouse (IgG2a) anti-human CD3), BMA030 (mouse (IgG2a) anti-human CD3), YTH 12.5 (rat (IgG2b) anti-human CD3), and NI-0401 (fully human anti-human CD3). Those of ordinary skill in the art will be aware of other anti-CD3 antibodies and antigen binding fragments thereof that can be used in accordance with the dosing regimens disclosed herein.

In certain embodiments, an anti-CD3 antibody or antigen binding fragment that exhibits reduced binding to at least one class of Fc (gamma) receptor is modified in that it lacks some or all of an Fc domain. For example, Fab fragments and F(ab′)₂ fragments lack some or all of an Fc domain.

In certain embodiments, an anti-CD3 antibody or antigen binding fragment is modified in some other way such that it exhibits reduced binding to at least one class of Fc (gamma) receptor. For example, the anti-CD3 antibody or antigen binding fragment may be modified by covalently linkage of a chemical moiety that prevents the anti-CD3 antibody or antigen binding fragment from binding at least one class of Fc (gamma) receptor. As another example, the anti-CD3 antibody or antigen binding fragment may be modified by non-covalently linkage of a chemical moiety that prevents the anti-CD3 antibody or antigen binding fragment from binding at least one class of Fc (gamma) receptor. Any of a variety of moieties may be covalently or non-covalently linked to the anti-CD3 antibody or antigen binding fragment to prevent binding to at least one class of Fc (gamma) receptor. Those of ordinary skill in the art will be aware of suitable moieties that can be linked to an antibody or antigen binding fragment, and will be able to employ such moieties in accordance with the teachings herein.

Those of ordinary skill in the art will be aware of other anti-CD3 antibodies and fragments that exhibit reduced binding to at least one class of Fc (gamma) receptor, which antibodies and fragments can be employed in one or more of the dosing regimens disclosed herein.

### Pharmaceutical Formulations

Anti-CD3 antibodies or antigen binding fragments described herein may be formulated for delivery by any available route including, but not limited to parenteral (e.g., intravenous, intradermal, or subcutaneous), oral, nasal, bronchial, ophthalmic, transdermal (topical), transmucosal, rectal, and vaginal routes. The anti-CD3 antibody- or antigen binding fragment-containing compositions may include a delivery agent (e.g., a cationic polymer, peptide molecular transporter, surfactant, etc., as described above) and/or a pharmaceutically acceptable carrier. As used herein the term “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into pharmaceutical formulations comprises an anti-CD3 antibody or antigen binding fragment as described herein.

A pharmaceutical composition is formulated to be compatible with its intended route of administration. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injection or infusion typically include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be sterile and should be fluid to the extent that easy syringability exists. Pharmaceutical formulations are ideally stable under the conditions of manufacture and storage and should be preserved against the contaminating action of microorganisms such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be advantageous to include isotonic agents, for example, sugars, polyalcohols such as manitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the anti-CD3 antibody or antigen binding fragment in the required amount in an appropriate solvent with one or a combination of ingredients enumerated above, as required, followed by filtered sterilization. Generally, dispersions are prepared by incorporating the purified antibody or antigen binding fragment into a sterile vehicle which contains a basic dispersion medium and the required other ingredients from those enumerated above. In the case of sterile powders for the preparation of sterile injectable solutions, exemplary methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the anti-CD3 antibody or antigen binding fragment can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Formulations for oral delivery may advantageously incorporate agents to improve stability within the gastrointestinal tract and/or to enhance absorption.

For administration by inhalation, the anti-CD3 antibody or antigen binding fragment and a delivery agent are preferably delivered in the form of an aerosol spray from a pressured container or dispenser which contains a suitable propellant, e.g., a gas such as carbon dioxide, or a nebulizer. The present disclosure particularly contemplates delivery of the compositions using a nasal spray, inhaler, or other direct delivery to the upper and/or lower airway. Intranasal administration of DNA vaccines directed against influenza viruses has been shown to induce CD8+T cell responses, indicating that at least some cells in the respiratory tract can take up DNA when delivered by this route, and the delivery agents of the invention will enhance cellular uptake. According to certain embodiments, anti-CD3 antibody or antigen binding fragment and a delivery agent are formulated as large porous particles for aerosol administration.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the purified polypeptide or protein and delivery agents are formulated into ointments, salves, gels, or creams as generally known in the art.

In certain embodiments, compositions are prepared with carriers that will protect the anti-CD3 antibody or antigen binding fragment against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811, the disclosure of which is incorporated herein by reference in its entirety.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active anti-CD3 antibody or antigen binding fragment thereof calculated to produce the desired therapeutic effect in association with the required pharmaceutical carrier.

The anti-CD3 antibody or antigen binding fragment thereof can be administered at various intervals and over different periods of time as required, e.g., one time per week for between about 1 to 10 weeks, between 2 to 8 weeks, between about 3 to 7 weeks, about 4, 5, or 6 weeks, etc. Those of ordinary skill in the art will appreciate that certain factors can influence the dosage and timing required to effectively treat a subject, including but not limited to the severity of the disease or disorder, previous treatments, the general health and/or age of the subject, and other diseases present. Generally, treatment of a subject with an anti-CD3 antibody or antigen binding fragment as described herein can include a single treatment or, in many cases, can include a series of treatments. It is furthermore understood that appropriate doses may depend upon the potency of the anti-CD3 antibody or antigen binding fragment and may optionally be tailored to the particular recipient, for example, through administration of increasing doses until a preselected desired response is achieved. It is understood that the specific dose level for any particular animal subject may depend upon a variety of factors including the activity of the specific polypeptide or protein employed, the age, body weight, general health, gender, and diet of the subject, the time of administration, the route of administration, the rate of excretion, any drug combination, and the degree of expression or activity to be modulated.

Pharmaceutical formulations as described herein can be included in a container, pack, or dispenser together with instructions for administration.

### Combination Therapies

Anti-CD3 antibodies and antigen binding fragments thereof can be administered to decrease weight gain or increase weight loss in subject with BMIs greater than or equal to about 27 according to one or more dosing regimens disclosed herein, in combination with one or more other therapeutic agents. In certain embodiments, such a therapeutic agent works in combination (e.g., additively or synergistically) with the anti-CD3 antibody or fragment thereof to decrease weight gain or increase weight loss. Therapeutic agents that can be administered in combination with the anti-CD3 antibody or antigen binding fragment include, but are not limited to, peptides, polypeptides, conjugates, nucleic acid molecules (e.g., DNA or RNA), small molecules, mimetic agents, synthetic drugs, inorganic molecules, and organic molecules.

In certain embodiments, a therapeutic agent to be used in combination with the anti-CD3 antibody or antigen binding fragment is an immunomodulatory agent. In certain embodiments, such an immunomodulatory agent works in combination (e.g., additively or synergistically) with the anti-CD3 antibody or fragment thereof to decrease weight gain or increase weight loss. Any of a variety of immunomodulatory agent known to those of skill in the art may be administered in combination with the anti-CD3 antibody or antigen binding fragment, as disclosed herein. Immunomodulatory agents typically affect one or more aspects of an immune response in a subject including, without limitation, an inflammatory response, a complement cascade, leukocyte and lymphocyte differentiation, proliferation, and/or effector function, monocyte and/or basophil counts, and the cellular communication among cells of the immune system. Non-limiting examples of immunomodulatory agents include proteinaceous agents such as cytokines, peptide mimetics, and antibodies (e.g., human, humanized, chimeric, monoclonal, polyclonal, Fvs, scFvs, Fab or F(ab′)₂ fragments or epitope binding fragments), nucleic acid molecules (e.g., antisense nucleic acid molecules and triple helices), small molecules, organic compounds, and inorganic compounds. In particular, immunomodulatory agents include, but are not limited to, methotrexate, leflunomide, cyclophosphamide, cytoxan, Immuran, cyclosporine A, minocycline, azathioprine, antibiotics (e.g., FK506 (tacrolimus)), methylprednisolone (MP), corticosteroids, steroids, mycophenolate mofetil, rapamycin (sirolimus), mizoribine, deoxyspergualin, brequinar, malononitriloamindes (e.g., leflunamide). Other examples of immunomodulatory agents can be found, e.g., in United States Patent Publication Number 2005/0002934 A1 at paragraphs 259-275 which is incorporated herein by reference in its entirety. In certain embodiments, an immunomodulatory agent is a chemotherapeutic agent. In certain embodiments, an immunomodulatory agent is an immunomodulatory agent other than a chemotherapeutic agent.

In certain embodiments, a therapeutic agent to be used in combination with the anti-CD3 antibody or antigen binding fragment is a weight loss agent. For example, a weight loss agent can be a dietary supplement such as, but not limited to, ephedra, bitter orange, guarana, caffeine, country mallow, yerba maté, chromium, ginseng, guar gum, glucomannan, psyllium, 1-carnitine, hydroxycitric acid, green tea, vitamin B5, licorice, conjugated linoleic acid, pyruvate, chitosan, dandelion, cascara, St. John's wort, laminaria, spirulina (also known as blue-green algae), guggul, and/or apple cider vinegar. Other examples of suitable weight loss agents include without limitation, sibutramine and other serotonin and norepinephrine reuptake inhibitors (e.g., lorcaserin), orlistat (tetrahydrolipstatin), cetilistat, rimonabant, metformin, exenatide, and pramlintide.

In yet other embodiments, the anti-CD3 antibody or antigen binding fragment is administered to a patient who has undergone, is undergoing, or will undergo treatment with a non-pharmacological anti-obesity (weight loss) therapy or regimen, such as, for example, a diet, an exercise regimen, and/or surgery (e.g., gastric bypass surgery). Such patient may also, in addition, undergo treatment with one of the additional therapeutic agents described above.

In certain embodiments, the anti-CD3 antibody or antigen binding fragment is administered in conjunction with at least one other therapeutic agent, e.g., a weight loss agent, such as one or more of those described above. For such combination therapy, the anti-CD3 antibody or antigen binding fragment can be administered to the same or different sites (i.e., via the same or different routes of administration), at the same or different times, for the same or different frequencies of administration, and in the same or different dosages than the at least one other therapeutic agent (e.g., weight loss agent).

Thus, in certain embodiments, a therapeutic agent to be used in combination with the anti-CD3 antibody or antigen binding fragment for increasing weight loss or decreasing weight gain is administered to a patient according to the same dosing regimen as the anti-CD3 antibody or antigen binding fragment. For example, if a particular dosing regimen calls for the anti-CD3 antibody or antigen binding fragment to be administered to a patient on five consecutive days, a therapeutic agent may also be administered to the patient on the same five consecutive days. The particular dose of the therapeutic agent to be administered can be chosen by those of ordinary skill in the art based on any of a variety of factors, including for example, that therapeutic agent's known effective dose, pharmacokinetic and/or pharmacodynamic interactions between the anti-CD3 antibody or antigen binding fragment and the therapeutic agent, and the like.

In other embodiments, a therapeutic agent to be used in combination with the anti-CD3 antibody or antigen binding fragment for increasing weight loss or decreasing weight gain is administered to a patient according to a different dosing regimen as the anti-CD3 antibody or antigen binding fragment. For example, if a particular dosing regimen calls for the anti-CD3 antibody or antigen binding fragment to be administered to a patient on five consecutive days, a therapeutic agent may also be administered to the patient on only one day, or on two, three, four, six, seven, eight or more consecutive days, or on non-consecutive days. Those of ordinary skill in the art will be aware of suitable dosing regimens for a given therapeutic agent and will be able to administer such a therapeutic agent to a patient according to that therapeutic agent's effective dosing regimen.

In certain embodiments, anti-CD3 antibodies and fragments thereof are administered to decrease weight gain or increase weight loss in subject with BMIs greater than or equal to about 27 according to one or more dosing regimens disclosed herein, in combination with a physical exercise regimen.

Certain embodiments of methods and compositions provided herein are further illustrated by the following examples. The examples are provided for illustrative purposes only. They are not to be construed as limiting the scope or content of the invention in any way.

### EXAMPLES

### Example 1

### Dosing Regimen CH1

Otelixizumab (also referred to herein as TRX4) is an anti-CD3 antibody having a humanized heavy chain (containing rat heavy chain variable (VH) CDRs 1, 2 and 3, four human VH framework regions, and a human IgG1 constant region), a chimeric light chain (containing a rat light chain variable region (VL) and a human light chain kappa constant region), and has an aglycosylated Fc region, in which Asn297 of SEQ ID NO: 1 has been mutated to A1a297. Residue numbers are given according to the Kabat canonical numbering system (see Kabat E A, Wu T T, Perry H, Gottesman K, and Foeller C. (1991) Sequences of Proteins of Immunological Interest, Fifth Edition. NIH Publication No. 91-3242, incorporated herein by reference in its entirety). Otelixizumab was administered intravenously to subjects according to the following dosing schedule: 0.1 mg on day 1, 0.2 mg on day 2, 0.3 mg on day 3, and 0.5 mg on days 4-8. Daily doses were administered approximately 24 hours apart, and each dose was administered by intravenous infusion over a course of about two hours. Subjects were weighed and body mass index (BMI) was determined at baseline (BL) immediately prior to treatment, and at twelve months following treatment. Results are shown in Table 2, below.

TABLE 2
Weight and BMI Data for Subject Administered CH1
CH1

| | | BL Weight | | |
|---|---|---|---|---|
| Subject | Weight (kg) | (kg) | Change (kg) | BL BMI |
| 045-0007 | 61.8 | 61.6 | 0.20 | 20.8 |
| 031-0005 | 64.4 | 63.5 | 0.86 | 21.9 |
| 038-0008 | 68.9 | 68.3 | 0.64 | 22.0 |
| 038-0005 | 77.7 | 76.2 | 1.50 | 23.4 |
| 016-0030 | 85.7 | 84.1 | 1.60 | 24.1 |
| 039-0014 | 69.4 | 72.3 | −2.90 | 25.7 |
| 039-0007 | 80.0 | 77.8 | 2.16 | 26.1 |
| 045-0003 | 73.5 | 74.0 | −0.52 | 27.7 |
| 038-0007* | 74.5 | 83.9 | −9.37 | 27.7 |
| 038-0006 | 101.6 | 99.3 | 2.31 | 33.3 |
| | ALL | MEAN (SEM) | −0.35 | (1.11) |
| | | N | 10 | |
| | | SD | 3.52 | |
| | BMI >= 27 | MEAN (SEM) | −2.53 | (3.52) |
| | | N | 3 | |
| | | SD | 6.09 | |

*subject did not receive all doses

As can be seen from Table 2, subjects with baseline BMIs greater than 27 that were administered the CH1 dosing regimen exhibited increased mean weight loss over twelve months compared to the mean weight loss of all the subjects in the study. Thus, subjects with baseline BMIs greater than 27 exhibited increased mean weight loss compared to subjects with baseline BMIs less than 27. Two subjects with baseline BMIs greater than 27 lost weight over the twelve month period.

### Example 2

### Dosing Regimen CH2

Otelixizumab was administered intravenously to a cohort of subjects diagnosed with Type I diabetes according to the following dosing schedule (“CH2”): 0.1 mg on day 1, 0.2 mg on day 2, 0.3 mg on day 3, and 0.75 mg on days 4-8. Daily doses were administered approximately 24 hours apart, and each dose was administered by intravenous infusion over a course of about two hours. Subjects were weighed and body mass index (BMI) was determined at baseline (BL) immediately prior to treatment, and at twelve months following treatment. Results are shown in Table 2, below.

TABLE 2
Weight and BMI Data for Subject Administered CH2
CH2

| | | BL Weight | | |
|---|---|---|---|---|
| Subject | Weight (kg) | (kg) | Change (kg) | BL BMI |
| 016-0035 | 55.4 | 55.1 | 0.30 | 18.5 |
| 038-0004 | 45.4 | 44.9 | 0.49 | 18.7 |
| 016-0036 | 56.2 | 58.3 | −2.10 | 19.3 |
| 029-0012 | 59.2 | 56.0 | 3.20 | 19.8 |
| 035-0034 | 79.4 | 79.7 | −0.30 | 20.0 |
| 046-0017* | 67.7 | 66.8 | 0.90 | 20.6 |
| 038-0003 | 62.1 | 60.3 | 1.81 | 21.1 |
| 016-8002 | 76.7 | 71.5 | 5.20 | 21.4 |
| 029-0011 | 64.6 | 63.9 | 0.70 | 21.4 |
| 046-0010 | 58.0 | 57.0 | 1.00 | 24.3 |
| 031-0007 | 82.1 | 75.8 | 6.35 | 24.3 |
| 031-0006 | 67.1 | 67.6 | −0.45 | 24.4 |
| 046-0015 | 77.7 | 82.5 | −4.80 | 24.6 |
| 046-0025* | 66.5 | 69.6 | −3.10 | 25.7 |
| 046-0008 | 86.3 | 83.8 | 2.50 | 26.5 |
| 029-0005 | 83.1 | 87.5 | −4.40 | 26.9 |
| 016-0031 | 82.5 | 85.5 | −3.00 | 27.0 |
| 046-0003 | 89.5 | 88.1 | 1.40 | 27.3 |
| 046-0001 | 90.1 | 91.5 | −1.40 | 29.2 |
| 046-0009 | 75.5 | 77.0 | −1.50 | 30.8 |
| 038-0009 | 89.5 | 101.6 | −12.11 | 32.1 |
| 035-0032 | 91.6 | 89.9 | 1.70 | 34.0 |
| 046-0007 | 111.8 | 96.7 | 15.10 | 34.9 |
| | ALL | MEAN (SEM) | 0.33 | (1.03) |
| | | N | 23 | |
| | | SD | 4.94 | |
| | BMI >= 27 | MEAN (SEM) | 0.03 | (3.06) |
| | | N | 7 | |
| | | SD | 8.089391886 | |

As can be seen from Table 2, subjects with baseline BMIs greater than or equal to 27 that were administered the CH2 dosing regimen exhibited less mean weight gain over twelve months compared to the mean weight gain of all the subjects in the study. Thus, subjects with baseline BMIs greater than 27 exhibited decreased mean weight gain compared to subjects with baseline BMIs less than 27. Four subjects with baseline BMIs greater than or equal to 27 lost weight over the twelve month period.

### Example 3

### Dosing Regimen CH3

Otelixizumab was administered intravenously to a cohort of 6 patients diagnosed with Type I diabetes according to the following dosing schedule (“CH3”): 0.1 mg on day 1, 0.2 mg on day 2, 0.3 mg on day 3, 0.75 mg on day 4, 1.0 mg on day 5, 1.25 mg on day 6, 1.5 mg on day 7, and 1.75 mg on day 8. Daily doses were administered approximately 24 hours apart, and each dose was administered by intravenous infusion over a course of about two hours. Subjects were weighed and body mass index (BMI) was determined at baseline (BL) immediately prior to treatment, and at twelve months following treatment. Results are shown in Table 3, below.

TABLE 3
Weight and BMI Data for Subject Administered CH3
COHORT 3

| | | BL Weight | | |
|---|---|---|---|---|
| Subject | Weight (kg) | (kg) | Change (kg) | BL BMI |
| 029-0006 | 59.9 | 57.4 | 2.52 | 19.28348 |
| 029-0007 | 64.9 | 63.7 | 1.17 | 19.77973 |
| 031-0008 | 61.0 | 61.2 | −0.23 | 21.46328 |
| 051-0002 | 67.8 | 65.9 | 1.90 | 22.56323 |
| 038-0010 | 89.8 | 89.4 | 0.41 | 26.73038 |
| | ALL | MEAN (SEM) | 1.15 | (0.49) |
| | | N | 5 | |
| | | SD | 1.10 | |

As can be seen from Table 3, each of the subjects that were administered the CH3 dosing regimen exhibited a baseline BMI of less than 27. Four of the five subjects exhibited weight gain over a twelve month period.

Otelixizumab is administered intravenously to a cohort of subjects diagnosed with Type I diabetes according to dosing regimen CH3, which subjects have baseline BMIs greater than or equal to 27. It is expected that subjects with a baseline BMI greater than or equal to 27 that are administered the CH3 dosing regimen exhibit less mean weight gain or increased mean weight loss over twelve months than subjects with baseline BMIs less than 27.

### Example 4

### Dosing Regimen CH4

Otelixizumab is administered intravenously to a cohort of patients diagnosed with Type I diabetes according to the following dosing schedule (“CH4”): 0.1 mg on day 1, 0.2 mg on day 2, 0.3 mg on day 3, 0.75 mg on day 4, 1.0 mg on day 5, 1.25 mg on day 6, 1.5 mg on day 7, and 3.75 mg on day 8. Daily doses are administered approximately 24 hours apart, and each dose is administered by intravenous infusion over a course of about two hours, except for the 3.75 mg dose on day eight which is administered over a course of about four hours. Subjects are weighed and body mass index (BMI) is determined at baseline (BL) immediately prior to treatment, and at twelve weeks following treatment.

It is expected that subjects with a baseline BMI greater than 27 that are administered the CH4 dosing regimen exhibit less mean weight gain or increased mean weight loss over twelve months than subjects with baseline BMIs less than 27.

### Example 5

### Dosing Regimen CH5

Otelixizumab is administered intravenously to a cohort of patients diagnosed with Type I diabetes according to the following dosing schedule (“CH5”): 0.2 mg on day 1, 0.4 mg on day 2, 0.6 mg on day 3, 0.8 mg on day 4, and 1.1 mg on day 5. Daily doses are administered approximately 24 hours apart, and each dose is administered by intravenous infusion over a course of between about fifteen minutes and about two hours. Subjects are weighed and body mass index (BMI) is determined at baseline (BL) immediately prior to treatment, and at twelve months following treatment.

It is expected that subjects with a baseline BMI greater than 27 that are administered the CH5 dosing regimen exhibit less mean weight gain or increased mean weight loss over twelve months than subjects with baseline BMIs less than 27.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of administering to a human an anti-CD3 antibody or anti-CD3 antigen binding fragment, the method comprising:
selecting a human with a body mass index greater than or equal to about 27 in a dosing regimen;
administering the antibody or antigen binding fragment to the selected human;
wherein the amount of the antibody or antigen binding fragment administered on the first day of the dosing regimen is less than about 0.5 mg;
wherein the antibody or antigen binding fragment does not bind or has reduced binding to at least one class of Fc (gamma) receptor compared to the IgG1 immunoglobulin molecule produced by the cell line ARH-77 deposited under ATCC catalog number CRL-1621.

2. The method of claim 1, wherein the human gains less weight or loses more weight after or during the dosing regimen than would be observed in a human with a BMI less than 27 when administered an equivalent amount of the antibody or antigen binding fragment according to the dosing regimen.

3. The method of claim 1, wherein the human gains less weight or loses more weight after or during the dosing regimen than would be observed in a human with a BMI greater than or equal to about 27 when the human is not administered the antibody or antigen binding fragment according to the dosing regimen.

4. The method of claim 1, wherein the human gains less weight or loses more weight at the end of twelve months.

5. The method of claim 1, wherein the human loses more weight after or during the dosing regimen than would be observed in a human with a BMI greater than or equal to about 27 when the human is not administered the antibody or antigen binding fragment according to the dosing regimen.

6. The method of claim 1, wherein the human gains less weight after or during the dosing regimen than would be observed in a human with a BMI greater than or equal to about 27 when the human is not administered the antibody or antigen binding fragment according to the dosing regimen.

7. The method of claim 1, wherein the human has a body mass index greater than or equal to about 30.

8. The method of claim 1, wherein the human has a body mass index greater than or equal to about 32.

9. The method of claim 1, wherein the human has a body mass index greater than or equal to about 34.

10. The method of claim 1, wherein the human suffers from an immune-related disease.

11. The method of claim 10, wherein the immune-related disease is selected from the group consisting of: type I diabetes, type II diabetes, psoriasis, rheumatoid arthritis, lupus, inflammatory bowel disease, ulcerative colitis, Crohn's disease, Graves' thyroiditis, Graves' ophthalmopathy, multiple sclerosis, metabolic syndrome, effects form organ transplantation, or graft-versus-host disease (GVHD).

12. The method of claim 10, wherein the immune-related disease is diabetes.

13. The method of claim 1, wherein the human does not suffer from an immune-related disease.

14. The method of claim 1, wherein the antigen binding fragment is selected from the group consisting of a Fab fragment, a F(ab′)₂ fragment and a scFv fragment.

15. The method of claim 1, wherein the antibody or antigen binding fragment is chimeric.

16. The method of claim 1, wherein the antibody or antigen binding fragment is humanized.

17. The method of claim 1, wherein the antibody or antigen binding fragment comprises an Fc domain, wherein the Fc domain is aglycosylated.

18. The method of claim 1, wherein the antibody or antigen binding fragment comprises an amino acid sequence of SEQ ID NO: 3.

19. The method of claim 1, wherein the antibody or antigen binding fragment comprises an amino acid sequence of SEQ ID NO: 4.

20. The method of claim 1, wherein the antibody or antigen binding fragment comprises an amino acid sequence of SEQ ID NO: 3, and further comprises an amino acid sequence of SEQ ID NO: 4.

21. The method of claim 1, wherein the antibody or antigen binding fragment comprises an alanine at an amino acid position corresponding to amino acid position 299 of SEQ ID NO: 1.

22. The method of claim 1, wherein the antibody is selected from the group consisting of hOKT3, hOKT3-γ1(Ala-Ala), HUM291, and NI-0401.

23. The method of claim 1, wherein the antibody or antigen binding fragment exhibits at least 50% reduced binding to at least one class of Fc (gamma) receptor compared to the IgG1 antibody deposited under ATCC accession number CRL-1621.

24. The method of claim 1, wherein the antibody or antigen binding fragment exhibits at least 50% reduced binding to at least one class of Fc (gamma) receptor compared to the OKT3 antibody.

25. The method of claim 1, wherein the dosing regimen is five days.

26. The method of claim 1, wherein the dosing regimen is eight days.

27.-55. (canceled)


SEQUENCE LISTING (10 sequences)

SEQ ID NO: 1 - protein, 449 aa, Artificial Sequence
  HUMANIZED MURINE ANTIBODY HEAVY CHAIN
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 216 aa, Artificial Sequence
  HUMANIZED MURINE ANTIBODY LIGHT CHAIN
DIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - protein, 119 aa, Artificial Sequence [sequence omitted]

SEQ ID NO: 4 - protein, 111 aa, Artificial Sequence
  HUMANIZED MURINE ANTIBODY LIGHT CHAIN VARIABLE REGION
DIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLR

SEQ ID NO: 5 - protein, 5 aa, Mus Musculus
SFPMA

SEQ ID NO: 6 - protein, 17 aa, Mus Musculus
TISTSGGRTYYRDSVKG

SEQ ID NO: 7 - protein, 10 aa, Mus Musculus
FRQYSGGFDY

SEQ ID NO: 8 - protein, 13 aa, Mus Musculus
TLSSGNIENNYVH

SEQ ID NO: 9 - protein, 7 aa, Mus Musculus
DDDKRPD

SEQ ID NO: 10 - protein, 9 aa, Mus Musculus
HSYVSSFNV